(19) United States
(12) Patent Application Publication
(10) Pub. No.: US 2019/0031613 A1
(43) Pub. Date: Jan. 31, 2019

(54) COMPOUNDS AND METHODS FOR THEIR USE IN THE TREATMENT OF MALARIA

(71) Applicants: University of South Florida, Tampa, FL (US); Northeastern University, Boston, MA (US)

(72) Inventors: ROMAN MANETSCH, BOSTON, MA (US); DENNIS E. KYLE, LITHIA, FL (US); ANDRII MONASTYRSKYI, JUPITER, FL (US); ALEXIS N. LACRUE, TEMPLE TERRACE, FL (US); JORDANY R. MAIGNAN, LAND O LAKES, FL (US); FABIAN MARCEL BROCKMEYER, MALDEN, MA (US)

(21) Appl. No.: 16/072,088

(22) PCT Filed: Jan. 23, 2017
(86) PCT No.: PCT/US2017/014592

§ 371 Date: Jul. 23, 2018

Related U.S. Application Data
(60) Provisional application No. 62/281,819, filed on Jan. 22, 2016.

(51) Int. Cl. C07D 215/233; A61P 33/06

(52) U.S. Cl. CPC C07D 215/233; A61P 33/06

(57) ABSTRACT

Disclosed herein, in part, are compounds and methods for their use in the treatment of malaria. In at least one specific embodiment, the compounds or salts thereof can include compounds of Formula (I):

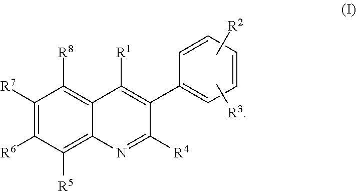

### BACKGROUND OF THE INVENTION

Malaria remains one of the most devastating parasitic diseases, with approximately 200 million reported infections and over 0.6 million of deaths per year.¹ While malaria is an entirely preventable and treatable mosquito-borne illness, children under the age of five account for almost 80% of the documented deaths. Five species of the genus Plasmodium (P. falciparum, P. vivax, P. ovale, P. malariae, and P. knowlesi) are responsible for malaria in humans, of which P. falciparum and P. vivax cause the majority of severe malaria cases. Recently, a decline in malaria morbidity and mortality has been observed as a result of combined efforts in preventing, controlling and treating malaria worldwide.² Nevertheless, commonly used antimalarials lose potency at an alarming rate due to widespread prevalence of drug resistant parasites. For example, resistance to chloroquine, one of the most commonly used antimalarials, has been confirmed in nearly all regions affected by malaria.³ Artemisinin combination therapies (ACTs) have arisen to combat malaria resistant to traditional medicines, and presently serve as a last-resort treatment. Unfortunately, a recent WHO report indicates that resistance to artemisinin has emerged at least in five countries of South-East Asia.¹ Due to the limited number of antimalarial chemotypes and rising P. falciparum resistance to most available medicines, new drugs are urgently required to combat this deadly diseas.^{4, 5}

Antimalarial drug discovery mostly focuses on the erythrocytic stages of malaria, which cause the disease. In order to combat the pernicious problem of parasitic resistance, it would benefit the community to develop agents capable of blocking multiple stages of the parasite life cycle. The best-known antimalarials that kill dormant liver stages and gametocytes are the 8-aminoquinolines primaquine and tafenoquine, developed more than 20 years ago.^{6, 7, 8} Unfortunately, both compounds cause hemolysis in individuals with a glucose-6-phosphate dehydrogenase deficiency (an estimated 400 million people worldwide).⁹ To guide the development of new antimalarials, the Malaria Eradication Research Agenda (malERA) initiative defined Target Product Profiles (TPPs) for antimalarial drugs to treat and prevent malaria infections, or to be used for radical cures of P. falciparum or P. vivax.¹⁰ These TPPs list important benchmark criteria, such as potent activity against resistant parasites, good oral bioavailability, a specific mechanism of action to effectively target multiple stages of the parasite life cycle, a shelf life of 5 years, low costs of active ingredients and formulations in the final medicine, and others.^{10, 11} An ambitious Single Exposure Radical Cure and Prophylaxis (SERCaP) treatment would require the ideal drug to be potent enough to work in a single, curative dose to treat P. falciparum and P. vivax infections.^{11, 12} A curative dose, in this context, is one which eliminates all persistent blood-stages, gametocytes and hypnozoites of the parasite. The antimalarials currently in clinical trials, ozonide OZ429,¹³ aminopyri dine MMV390048,^{14, 15} 3,4-dihydro-1(2H)-isoquinolone (+)-SJ733,¹⁶ spiroindolone KAE609¹⁷ and triazolopyrimidine DSM265¹⁸ have been reported to be a part of a single exposure radical cure initiative (PO dose 20 mg/kg for OZ429, 30 mg/kg for MMV390048, 100 mg/kg for KAE609, lowest single-cure dose data has not been reported for (+)-SJ733 and DSM265).

Recent evaluation and optimization studies of antimalarial 4(1H)-quinolones,^{19, 20, 21, 22, 23, 24} 4(1H)-pyridones,²⁵ 1,2,3,4-tetrahydroacridones,²⁶ 4(1H)-quinolone esters,^{27, 28, 29} and 2-aryl-4(1H)-quindlones³⁰ led to new agents with potent in vitro and in vivo erythrocytic stage activity and improved physicochemical properties.³¹ Extensive development of the 3-aryl-4(1H)-quinolone³² chemotype series resulted in frontrunner compound ELQ-300 (1a) and its close analog P4Q-391 (1b).³³ The chemical structures of compounds ELQ-300, P4Q-391, 1a, and 1b are shown below:

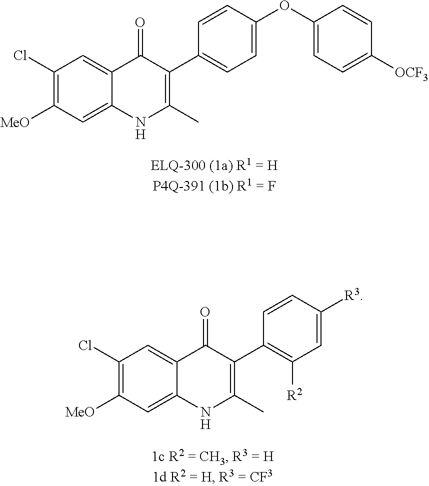

These compounds are potent and selective inhibitors of the parasite's mitochondrial cytochrome bc₁ complex and efficiently target the blood stages, the liver stages, and the transmitting stages of the parasite in murine models. The potent blood stage activity and demonstrated potential to kill hypnozoites in the gold standard P. cynomolgi infected Rhesus model make them attractive compounds to treat multidrug resistant malaria, to eradicate exoerythrocytic (EE) stages, to block transmission, and to aid in the malaria elimination campaign. Spearheaded by the Medicines for Malaria Venture, ELQ-300 (1a) entered preclinical development in 2013. Unfortunately, the advancement of 1a towards Phase I studies was deferred due to poor oral bioavailability, limiting preclinical safety and toxicity studies. Moreover, the absence of dose-proportionality impeded the determination of therapeutic index and in vivo toxicity.³³ DMPK. studies with lead quinolone compounds suggested aqueous solubility to be the major reason for poor oral bioavailability in the series.³⁴

The conversion of 4(1H)-quinolone 1a into an ethylcarbonate prodrug, utilizing the reactivity of the hydroxy group of the respective tautomeric 4-quinolinol 1a′, was recently described by Riscoe and co-workers.³⁴ One of the major disadvantages of this approach, which may complicate the development, is the reliance of the carbonate prodrug on esterases for the release of the parent drug.³⁵ For example, differences between specific esterase activities in various animal models possibly complicate dosing predictions for in vivo efficacy and pharmacokinetics in humans.³⁶ More importantly, the reported carbonate prodrug of 1a required a dissolution step using neat PEG 400 prior to performing in vivo efficacy and PK studies.³⁴

Due to the limited number of antimalarial chemotypes and rising P. falciparum resistance to most available medicines, new drugs are urgently required to combat this deadly disease.4, 5

### BRIEF SUMMARY OF THE INVENTION

Provided herein are new compounds and methods of using the same in the treatment malaria. In at least one specific embodiment, the compound or a salt thereof, can include compounds of Formula (I):

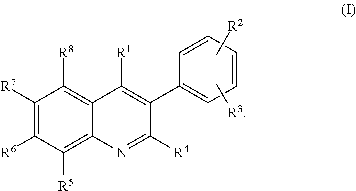

In another specific mbodiment, the method can include administering a composition that includes one or more compounds of Formula (I).

### BRIEF DESCRIPTION OF THE DRAWINGS

In the following detailed description, reference is made to the accompanying figures, depicting exemplary, non-limiting and non-exhaustive embodiments of the invention. So that the manner in which the above recited features of the present invention can be understood in detail, a more particular description of the invention, briefly summarized above, can be had by reference to the embodiments, some of which are illustrated in the appended figures. It should be noted, however, that the appended figures illustrate only typical embodiments of this invention and are therefore not to be considered limiting of its scope, for the invention can admit to other equally effective embodiments.

FIG. 1 shows the prodrug strategies for 4(1H)-quinolones 1.

FIG. 2a depicts the synthesis of 4(1H)-quinolones 1 via Conrad-Limpach reaction²⁴ and conversion to first set of prodrugs (2, 3 and 4) via O-acylation. FIG. 2b shows the results of in vitro antimalarial activities and solubility of k and prodrugs 2, 3 and 4, Chloroquine (CQ), at vaquone (ATO) and dihydroartemisinin (DHA) are internal controls for each in vitro assay: CQ, 421 nM W2, 229 nM TM90-C2B; ATO, 1.4 nM W2, 18.4 μM TM90-C2B; DHA, 5.5 nM W2, 5 nM TM90-C2B.

FIG. 3a depicts a proposed mechanism of the pH-activated parent compound release of amino AOCOM (alkoxyca,rbonyloxymethyl) prodrugs via an intramolecular nucleophilic attack. FIG. 3b shows a plot of the in vitro parent compound release profiles of 4(1H)-quinolone amino AOCOM ether prodrugs 6c at pH 2.0, 4.0, 7.0, SGF (simulated gastric fluid; pH˜1.2) and SIF (simulated intestinal fluid; pH˜6.5). FIG. 3c shows a plot of an the in vitro parent compound release profiles of 4(1H)-quinolone amino AOCOM ether prodrugs 6d at pH 2.0, 4.0, 7.0, SGF (simulated gastric fluid; pH˜1.2) and SIF (simulated intestinal fluid; pH˜6.5). The stability of prodrugs in aqueous media and the release of parent compound was followed for 10 h using HPLC in triplicates.

FIG. 4a depicts the synthesis of 4(1H)-quinolones 1 into amino AOCOM ether prodrugs 6 via O-alkylation and subsequent deprotection. FIG. 4b shows a plot of the in vitro antimalarial activities and solubility of amino AOCOM ether prodrugs 6. Chloroquine (CQ), atovaquone (ATO) and dihydroartemisinin (DHA) are internal controls for each in vitro assay: CQ, 421 nM W2, 229 nM TM90-C2B; ATO, 1.4 nM W2, 18.4 μM TM90-C2B; DHA, 5.5 nM W2, 5.9 nM TM90-C2B.

FIG. 5a shows the plasma concentration of 4(1H)-quinolone 1d after single oral administration of 50 mg/kg of 1d and corresponding amino AOCOM ether prodrugs 6c and 6d in 0.5% aqueous HEC formulation, FIG. 1b shows the plasma concentration of 4(1H)-quinolone 1b after single oral administration of 1b and corresponding amino AOCOM ether prodrug 6e in 0.5% aqueous HEC formulation.

FIG. 6 show a plot of the dose-linearity graph

FIG. 7 shows the ¹H NMR spectrum of S1a.

FIG. 8 shows the ¹³C NMR spectrum of S1a.

FIG. 9 shows the ¹H NMR spectrum of S1b.

FIG. 10 shows the ¹³C NMR spectrum of S1b.

FIG. 11 shows the ¹H NMR, spectrum of S2a.

FIG. 12 shows the ¹³C NMR spectrum of S2a.

FIG. 13 shows the ¹H NMR spectrum of S2b.

FIG. 14 shows the ¹³C NMR spectrum of S2b.

FIG. 15 shows the ¹H NMR spectrum of 2a,

FIG. 16 shows the ¹³C NMR spectrum of 2a.

FIG. 17 shows the ¹H NMR. spectrum of 2b.

FIG. 18 shows the ¹³C NMR spectrum of 2b.

FIG. 19 shows the ¹H NMR spectrum of 3.

FIG. 20 shows the ¹³C NMR spectrumof 3.

FIG. 21 shows the ¹H NIVIR spectrum of 4.

FIG. 22 shows the ¹³C NMR spectrum of 4.

FIG. 23 shows the ¹H NMR spectrum of 5a.

FIG. 24 shows the ¹³C NMR spectrum of 5a.

FIG. 25 shows the ¹H NMR spectrum of 5b.

FIG. 26 shows the ¹³C NMR spectrum of 5b.

FIG. 27 shows the ¹H NMR spectrum of 5c.

FIG. 28 shows the ¹³C NMR spectrum of 5c.

FIG. 29 shows the ¹H NMR spectrum of 5d.

FIG. 30 shows the ¹³C NMR spectrum of 5d.

FIG. 31 shows the ¹H NMR spectrum of 5e.

FIG. 32 shows the ¹³C NMR spectrum of 5e.

FIG. 33 shows the ¹H/¹³C HSQC NMR spectrum of 5e.

FIG. 34 shows the ¹H/¹³C HMBC NMR spectrum of 5e.

FIG. 35 shows the ¹H NMR spectrum of 6a.

FIG. 36 shows the ¹³C NMR spectrum of 6a.

FIG. 37 shows the ¹H NMR spectrum of 6b.

FIG. 38 shows the ¹³C NMR spectrum of 6b.

FIG. 39 shows the ¹H NMR. spectrum of 6c.

FIG. 40 shows the ¹³C NMR spectrum of 6c.

FIG. 41 shows the ¹H NMR spectrum of 6d.

FIG. 42 shows the ¹³C NMR spectrum of 6d.

FIG. 43 shows the ¹H NMR spectrum of 6e.

FIG. 44 shows the ¹³C NMR spectrum of 6e.

FIG. 45 shows the ¹H/¹³C HSQC NMR spectrum of 6e.

FIG. 46 shows the ¹H/¹³C HMBC NMR spectrum of 6e.

FIG. 47 shows P4Q-146 and P4Q-158 and their prodrugs' in vivo efficacy.

FIG. 48 shows P4Q-158 and its prodrugs' pharmacokinetic profiles.

FIG. 49 shows P4Q-391 and its prodrug's in vivo efficacy.

### DETAILED DISCLOSURE OF THE INVENTION

The compounds can include, but are not limited to, 4(1H)-quinolone derivatives. The compounds can be used in a prodrug approach in the treatment of malaria. Moreover, the compounds can ameliorate the oral bioavailability limitations of other drugs. For example, the in vivo efficacy was significantly improved with prodrugs of 4(1H)-quinolone-based antimalarials ICI 56,780, WR 243246 and P4Q-391, thereby, proving the versatility and applicability of a prodrug approach to any 4(1H)-quinolone scaffold with limited oral bioavailability. Surprisingly, the prodrug of 3-aryl-substituted 4(1H)-quinolone, P4Q-391, completely cured P. bergei-infected mice by a single oral dose of 3 mg/kg without the use of advanced formulations.

Also provided is a design and development of a prodrug approach to increase the aqueous solubility and the rate of dissolution of 4(1H)-quinolone leads, which improves the oral bioavailability commonly observed for 4(1H)-quinolones. Delivery of lead 4(1H)-quinolone compound 1b from its prodrug was enhanced 18-fold (relative to the administration of the parent 4(1H)-quinolone), reaching a Cₘₐₓ of 9.1 μM in 4 h following oral administration (single dose, 10 mg/kg). The developed prodrug approach was also successfully applied to other 4(1H)-quinolone-based antimalarials; thereby, proving the versatility and applicability of a prodrug approach to any 4(1H)-quinolone scaffold with limited oral bioavailability.

The compounds can include compounds of Formula

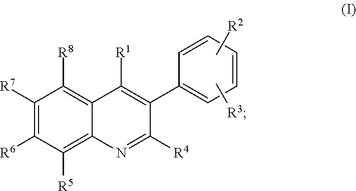

where R¹ is selected from H, F, Cl, Br, I, CN, CH₃, CF₃, hydroxyl, alkyl, halogenated alkyl, heteroalkyl, alkynyl, aryl, arylalkyl, aryloxy, arylalkoxy, heteroalkyl, heteroaryl, heterocyclyl, cycloalkyl, cycloalkenyl, cycloalkynyl, hydroxyalkyl, alkoxy, alkoxyalkyl, amino, aminoallkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), suifonate ester, sulfonamide, carbamate, alk-yltriphenylphosphonium,

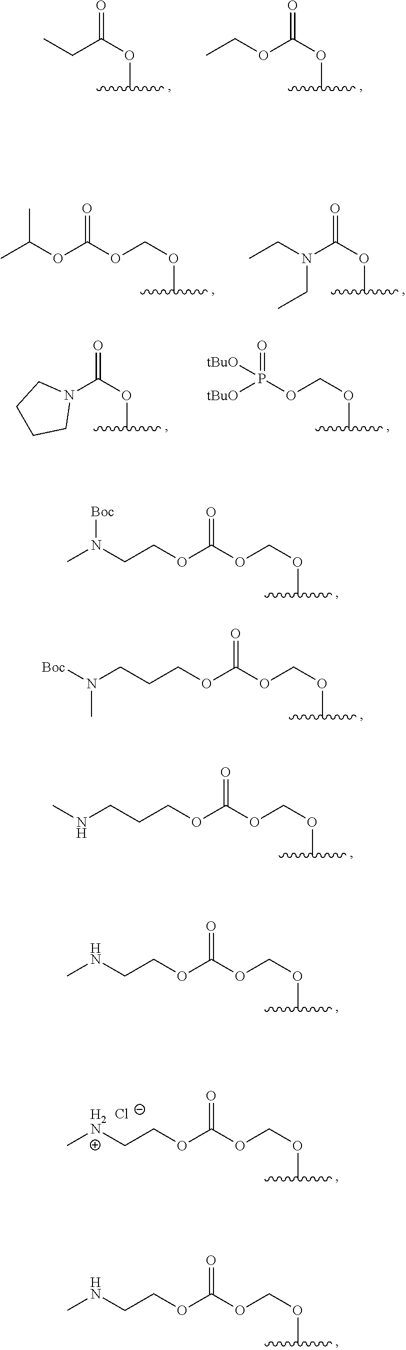

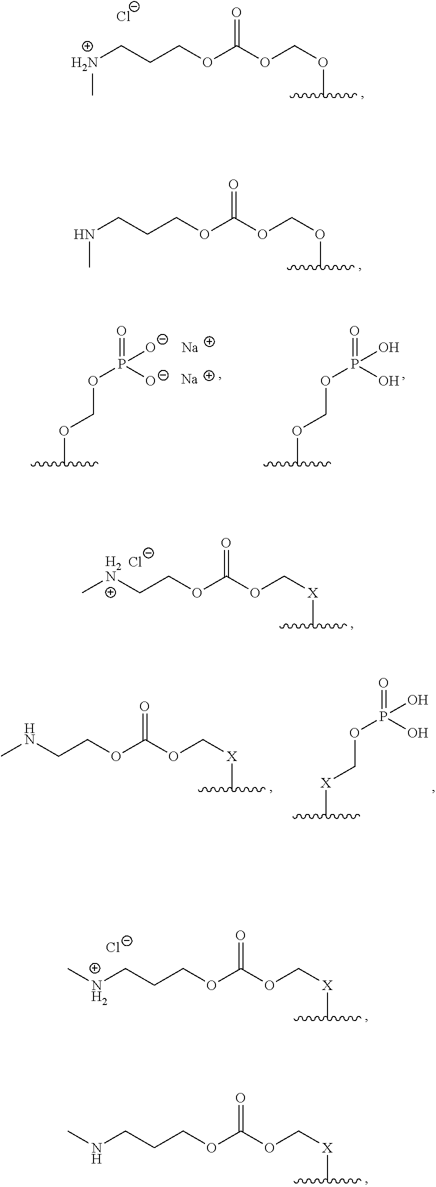

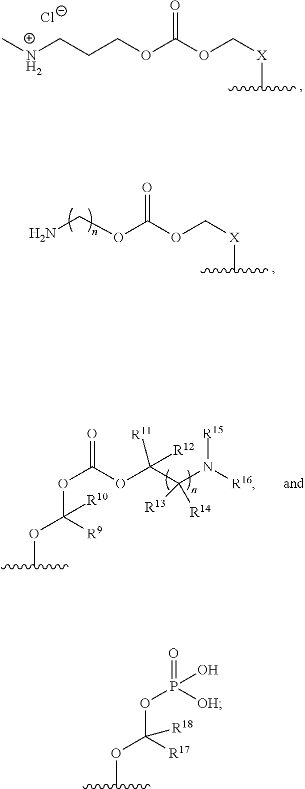

R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently selected from H, F, Cl, Br, I, CN, CH₃, CF₃, OCH₃, hydroxyl, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (SO₃H), sulfonate ester, sulfonamide, and carbamate, alkyltriphenylphosphoniutn, and

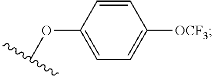

where X is selected from NH, NR¹⁹, oxygen, sulfur, and selenium, where R¹⁹ is selected from the group H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloa.lkyl, cycloalkenyl, cycloalkenyl, hydroxyl, hydroxyalkyl, al koxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium; and

where n is 1, 2, or 4.

As used herein, the term “alkyl” includes saturated aliphatic hydrocarbons including straight chains and branched chains. In some embodiments, the alkyl group has 1 to 20 carbon atoms, 1 to 10 carbon atoms, 1 to 6 carbon atoms, or 1 to 4 carbon atoms. For example, the term “C₁₋₆ alkyl,” as well as the alkyl moieties of other groups referred to herein (e.g., C₁₋₆ alkoxy) rerefers to linear or branched radicals of 1 to 6 carbon atoms (e.g., methyl, ethyl, n-propyl, isopropyl, n-butyl, isobutyl, sec-butyl, test-butyl, n-pentyl, or n-hexyl). For yet another example, the term “C₁₋₄ alkyl” refers to linear or branched aliphatic hydrocarbon chains of 1 to 4 carbon atoms; the term “C₁₋₃ alkyl” refers to linear or branched aliphatic hydrocarbon chains off to 3 carbon atoms; the term “C₁₋₂ alkyl” refers to linear or branched aliphatic hydrocarbon chains of 1 to 2 carbon atoms; and the term “C₁ alkyl” refers to methyl. The term “lower alkyl” refers to linear or branched radicals of 1 to 6 carbon atoms. An alkyl group optionally can be substituted by one or more (e.g. 1 to 5) suitable substituents.

As used herein, the term “alkenyl” includes aliphatic hydrocarbons haying at least one carbon carbon double bond, including straight chains and branched chains having at least one carbon-carbon double bond. In some embodiments, the alkenyl group has 2 to 20 carbon atoms. 2 to 10 carbon atoms, 2 to 6 carbon atoms, 3 to 6 carbon atoms, or 2 to 4 carbon atoms. For example, as used herein, the term “C₂₋₆ alkenyl” means straight or branched chain unsaturated radicals (haying at least one carbon-carbon double bond) of 2 to 6 carbon atoms, including, but not limited to, ethenyl, 1-propenyl, 2-propenyl (allyl), isopropenyl, 2-methyl-1-propenyl, 1-butenyl, 2-butenyl, and the like. An alkenyl group optionally can be substituted by one or more . 1 to 5 suitable substituents. When the compounds contain an alkenyl group, the alkenyl group may exist as the pure E form, the pure Z form, or any mixture thereof.

As used herein, the term “alkynyl” includes to aliphatic hydrocarbons having at least one carbon-carbon triple bond, including straight chains and branched chains having at least one carbon carbon triple bond. In some embodiments, the alkynyl group has 2 to 20, 2 to 10, 2 to 6, or 3 to 6 carbon atoms. For example, as used herein, the term “C2.₆ alkynyl” refers to straight or branched hydrocarbon chain alkynyl radicals as defined above, having 2 to 6 carbon atoms. An alkynyl group optionally can be substituted by one or more (e.g. 1 to 5) suitable substituents.

As used herein, the term “cycloalkyl” includes saturated or unsaturated, non-aromatic, monocyclic or polycyclic (such as bicyclic) hydrocarbon rings (e.g., monocyclics such as cyclopropyl, cyclobutyl, cyclopentyl, cyclohexyl, cycloheptyl, cyclooctyl, cyclononyl, or bicyclics including spiro, fused, or bridged systems (such as bicyclo[1.1.1]pentanyl, bicyclo[2.2.1]heptanyl, bicyclo[3.1.1]octanyl or bicyclo[5.2.0]nonanyl, decahydronaphthalenyl, etc.). The cycloalkyl group has 3 to 15 carbon atoms. In some embodiments the cycloalkyl may optionally contain one, two or more noncumulative non-aromatic double or triple bonds and/or one to three oxo groups. In some embodiments, the bicycloalkyl group has 6 to 14 carbon atoms. For example, the term “C₃₋₁₄ cycloalkyl” includes saturated or unsaturated, non-aromatic, monocyclic or polycyclic (such as bicyclic) hydrocarbon rings of 3 to 14 ring-forming carbon atoms (e.g., cyclopropyl, cyclobutyl, cyclopentyl, cyclohexyl, bicyclo[1.1.1]pentanyl, or cyclodecanyl); and the term “C₃₋₇ cycloalkyl” includes saturated or unsaturated, nonaromatic, monocyclic or polycyclic (such as bicyclic) hydrocarbon rings of 3 to 7 ring forming carbon atoms (e.g., cyclopropyl, cyclobutyl, cyclopentyl, cyclohexyl, bicyclo[1.1.1]pentan-1-yl, or bicyclo[1.1.1]pentan-2-yl). For another example, the term “C₃₋₆ cycloalkyl” includes saturated or unsaturated, non-aromatic, monocyclic or polycyclic (such as bicyclic) hydrocarbon rings of 3 to 6 ring-forming carbon atoms. For yet another example, the term “C₃₋₄ cycloalkyl” refers to cyclopropyl or cyclobutyl. Also included in the term “cycloalkyl” are moieties that have one or more aromatic rings (including aryl and heteroaryl) fused to the cycloalkyl ring, for example, benzo or thienyl derivatives of cyclopentane, cyclopentene, cyclohexane, and the like (e.g., 2,3-dihydro-1H-indene-1-yl, or 1H-inden-2(3H)-one-1-yl). The cycloalkyl group optionally can be substituted by 1 or more (e.g., 1 to 5) suitable substituents.

As used herein, the term “aryl” can include all-carbon monocyclic or fused-ring polycyclic aromatic groups having a conjugated pi-electron system. The aryl group has 6 or 10 carbon atoms in the ring(s). Most commonly, the aryl group has 6 carbon atoms in the ring. For example, as used herein, the term “C₆₋₁₀ aryl” means aromatic radicals containing from 6 to 10 carbon atoms such as phenyl or naphthyl. The aryl group optionally can be substituted by 1 or more (e.g., 1 to 5) suitable substituents. The term “arylene” refers to a divalent aryl moiety.

As used herein, the term “heteroaryl” includes monocyclic or fused-ring polycyclic aromatic heterocyclic groups with one or more heteroatom ring members (ring forming atoms) each independently selected from O, S and N in at least one ring. The heteroaryl group has 5 to 14 ring forming atoms, including 1 to 13 carbon atoms, and 1 to 8 heteroatoms selected from 0, S, and N. In some embodiments, the heteroaryl group has 5 to 10 ring-forming atoms including one to four heteroatoms. The heteroaryl group can also contain one to three oxo or thiono (i.e ═S) groups. In some embodiments, the heteroaryl group has 5 to 8 ring forming atoms including one, two or three heteroatoms. For example, the term “5-memberedheteroaryl” refers to a monocyclic heteroaryl group as defined above with 5 ring-forming atoms in the monocyclic heteroaryl ring; the term “6-membered heteroaryl” includes to a monocyclic heteroaryl group as defined above with 6 ring-forming atoms in the monocyclic heteroaryl ring; and the term “5- or 6-membered heteroaryl” includes a monocyclic heteroaryl group as defined above with 5 or 6 ring-forming atoms in the monocyclic heteroaryl ring. For another example, term “5- or 10-membered heteroaryl” includes a monocyclic or bicyclic heteroaryl group as defined above with 5, 6, 7, 8, 9 or 10 ring-forming atoms in the monocyclic or bicyclic heteroaryl ring. A heteroaryl group optionally can be substituted by 1 or more (e.g., 1 to 5) suitable substituents. Examples of monocyclic heteroaryls include those with 5 ring-forming atoms including one to three heteroatoms or those with 6 ring-forming atoms including one; two or three nitrogen heteroatoms. Examples of fused bicyclic heteroaryls include two fused 5- and/or 6-membered monocyclic rings including one to four heteroatoms.

As used herein, the term “heterocyclyl” includes saturated and partially saturated heteroatom-containing ring-shaped radicals having from 5 through 15 ring members selected from carbon, nitrogen, sulfur and oxygen, wherein at least one ring atom is a heteroatom. Heterocyclyl radicals may contain one, two or three rings wherein such rings may be attached in a pendant manner or may be fused. Examples of saturated heterocyclic radicals include saturated 3 to 6-membered heteromonocylic group containing 1 to 4 nitrogen atoms [e.g. pyrrolidinyl, piperidino, piperazinyl, etc.]; saturated 3 to 6-membered heterotnonocyclic group containing 1 to 2 oxygen atoms and 1 to 3 nitrogen atorris [e.g. morpholinyl, etc.]; saturated 3 to 6-membered heteromonocyclic group containing 1 to 2 sulfur atoms and 1 to 3 nitrogen atoms [e.g., thiazolidinyl, etc.]. Examples of partially saturated heterocyclyl radicals include dihydrothiophene, dihydropyran, dihydrofuran and dihydrothiazole. lion-limiting examples of heterocyclic radicals include 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like.

As used herein, the term “alkoxy” or “alkyloxy” include an —O-alkyl group. For example, the term “C₁₋₆ alkoxy” or “C₁₋₆ alkyloxy” includes an —O—(C₁₋₆ alkyl) group; and the term “C₁₋₄ alkoxy” or “C₁₋₄ alkyloxy” can include an —O—(C₁₋₄ alkyl) group. For another example, the term “C₁₋₂ alkoxy” or “C₁₋₂ alkyloxy” refers to an —O—(C₁₋₂ alkyl) group. Examples of alkoxy include methoxy, ethoxy, propoxy n-propoxy and isopropoxy), tert-butoxy, and the like. The alkoxy or alkyloxy group optionally can be substituted by 1 or more (e.g., 1 to suitable substituents.

As used here, the term “C₆₋₁₀ aryloxy” includes an —O—(C₆₋₁₀ aryl) group. An example of a C₆₋₁₀ aryloxy group is —O-phenyl [i.e., phenoxy]. The C₆₋₁₀ aryloxy group optionally can be substituted by 1 or more (e.g., 1 to 5) suitable substituents.

As used herein, the term “aminoalkyl” includes linear and/or branched alkyl radicals having one to about ten carbon atoms any one of which may be substituted with one or more amino radicals. Examples of such radicals include aminomethyl, aminoethyl, aminopropyl, aminobutyl and aminohexyl.

As used herein, the term “oxo” refers to ═O. When an oxo is substituted on a carbon atom, they together form a carbonyl moiety [—C(═O)—]. When an oxo is substituted on a sulfur atom, they together form a sulfonyl moiety [—S(═O)—]; when two oxo groups are substituted on a sulfur atom, they together form a sulfonyl moiety [—S(═O)₂—].

As used herein, the term “optionally substituted” means that substitution is optional and therefore includes both uns bstituted and substituted atoms and moieties. A “substituted” atom or moiety indicates that any hydrogen on the designated atom or moiety can be replaced with a selection from the indicated substituent group (up to that every hydrogen atom on the designated atom or moiety is replaced with a selection from the indicated substituent group), provided that the normal valency of the designated atom or moiety is not exceeded, and that the substitution results in a stable compound. For example, if a methyl group (i.e., CH₃) is optionally substituted, then up to 3 hydrogen atoms on the carbon atom can be replaced with substituent groups.

The method of treating malaria can include, but is not limited to, administering a composition that includes one or more compounds of Formula (I). The administration can include, but is not limited to: administration though oral pathways, which administration includes administration in capsule, tablet, granule, spray, syrup, or other such forms; administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, or the like; as well as administration topically; and administration via controlled released formulations, depot formulations, and infusion pump delivery. As further examples of such modes of administration and as further disclosure of modes of administration, disclosed herein are various methods for administration of the disclosed compounds and pharmaceutical compositions including modes of administration through intraocular, intranasal, and intraaudcular pathways.

In practicing the methods, the compounds of Formula (I) or the compositions can be used alone or in combination with one another, or in combination with other therapeutic or diagnostic agents. These products can be utilized in vivo, ordinarily in a mammal, preferably in a human, or in vitro. In employing them in vivo, the products or compositions can be administered to the mammal in a variety of ways, including parenterally, intravenously, subcutaneously, intramuscularly, colonically, rectally, vaginally, nasally or intraperitoneally, employing a variety of dosage forms. Such methods can also be applied to testing chemical activity in vivo.

The compounds of Formula (I) can be in the form of pharmaceutically acceptable salts. The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, sr bstituted amines including naturally occurring substituted amines, cyclic amines, basic ion exchange resins, and the like, specifically such as isopropylamine, trimethylamine, diethylamine triethylamine, tripropylamine, and ethanolamine.

The composition can include, but is not limited to, a pharmaceutically acceptable carrier. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the invention is contemplated. Supplementary active ingredients can also be incorporated into the compositions.

Determining a therapeutically effective amount of the composition is well within the capability of those skilled in the art. A “therapeutically effective amount” means that amount of the compound in the composition which, when administered to a human suffering from a malaria, is sufficient to effect treatment for the malaria.

A dose of a compound of Formula (I) can vary widely. For example, a dose of a compound of Formula (I) can be from a small of about 0.01 mg/kg, about 1 mg/kg, or about 2 mg/kg, to a large of about 4 mg/kg, about 7 mg/kg, or about 30 mg/kg. For example, a dose compound of Formula (I) can be from about 0.01 mg/kg to about 1 mg/kg, about 0.1 mg/kg to about 0.5 mg/kg, about 0.5 mg/kg to about 2 mg/kg, about 1 mg/kg to about 3 mg/kg, about 1 mg/kg to about 30 mg/kg, about 1.5 mg/kg to about 3.5 mg/kg, about 2.5 mg/kg to about 3.5 mg/kg, about 2.6 mg/kg to about 5 mg/kg, about 5 mg/kg to about 13 mg/kg, about 6 mg/kg to about 15 mg/kg, about 10 mg/kg to about 25 mg/kg, or about 15 mg/kg to about 30 mg/kg.

The composition that includes one or more compounds of Formula (I) can be used to treat many kinds of malarial strains. For example, the composition that includes one or more compounds of Formula (I) can be used to treat malarial strains that include, but are not limited to, W2, TM90-C2A, and TM90-C2B.

### Materials and Methods

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

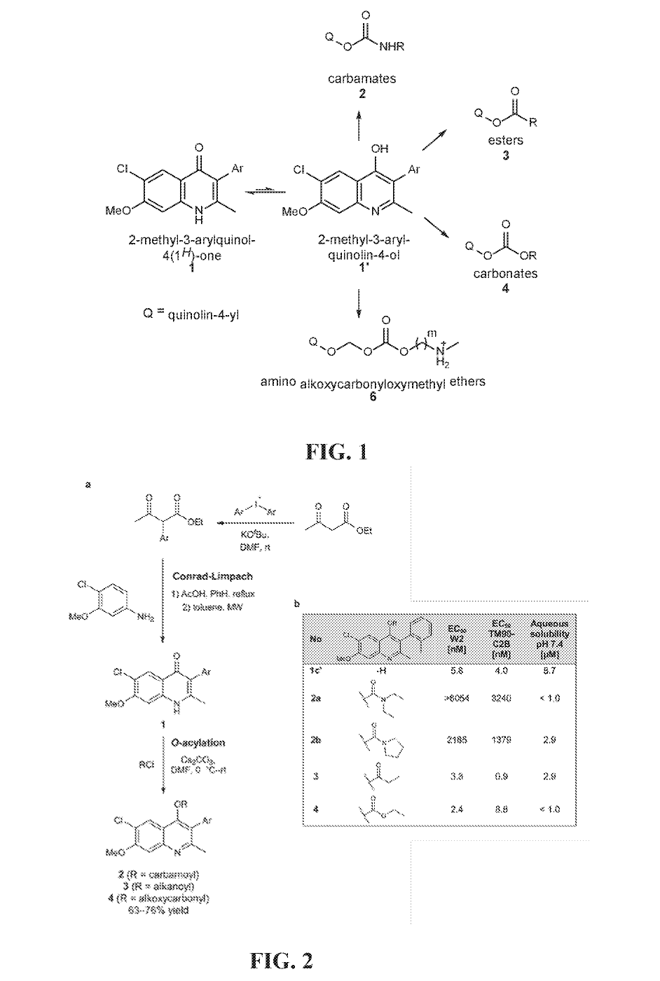

The development of a soluble quinolone prodrug begun by investigating the altered crystal packing of 4(1H)-quinolone prodrugs and determining its effect on aqueous solubility and dissolution rate. A series of easily accessible prodrugs, analogous to that reported by Riscoe and colleagues,³⁴ can be made by utilizing the hydroxy group of the tautomeric 4-quinolinols 1′ as the attachment site of the prodrug moiety as shown in FIG. 1. By linking the promoiety onto the oxygen of the 4-quinolinol 1′ this prodrug approach can be tailored to be applicable to any 4(1H)-quinolones independently of their core substitutions. Previous studies have shown that methylation of 4(1H)-quinolones provided mixtures of N- and C)-methylated products in a 2:1 ratio, whereas alkylations with larger alkyl halides yielded predominantly O-substituted 4-quinolinols^{20, 23, 32, 33, 37, 38} although the 4(1H)-quinolone tautomer is favored in both solid and solution states.³⁷

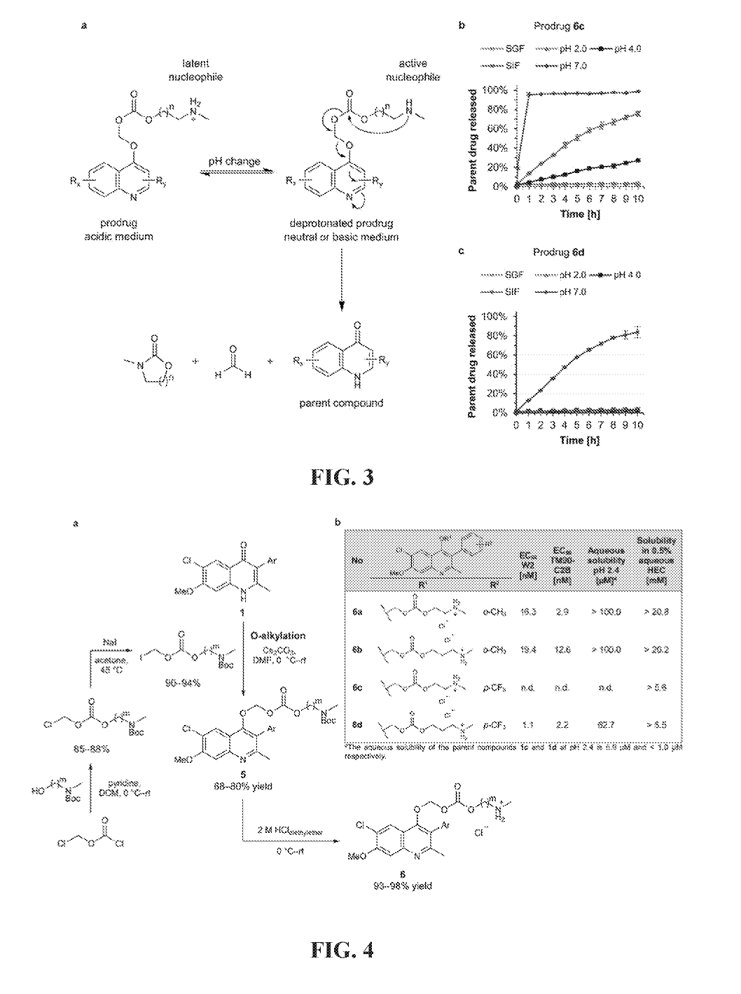

For investigation and optimization of a suitable promoiety, known 3-aryl-4(1H)-quinolones 1 were synthesized as previously described via a Conrad-Limpach cyclization of 2-aryl substituted ethyl acetoacetate with 3-chloro-4-methoxy aniline.^{20, 24} A first set of carbamate, ester and carbonate prodrugs 2, 3, and 4 (FIG. 2a) were obtained in moderate to good yields by derivatization of the 4-hydroxy group of 1c′ with corresponding electrophiles and caesium carbonate in DMF. 4(1H)-Quinolone 1c was chosen as the parent compound as it was shown to be slightly better soluble in comparison to the majority of 3-aryl-4(1H)-quinolones.³² All compounds were tested for in vitro activity against the clinically relevant multidrug resistant malarial strains W2 (pyrimethamine and chloroquine resistant strains) and TM90-C2B (mefloquine, chloroquine, atovaquone, pyrimethamine resistant strains) following previously reported procedures (FIG. 2b).^{27, 32} Aqueous solubility at pH 7.4 was determined using a previously reported protoco20, 26

In earlier studies, N- and O-methylated 4(1H)-quinolones displayed no antimalarial activity in vitro.²⁰ Similarly, carbamates 2a and 2b lacked in antimalarial activity with EC₅₀ values 300-fold higher than reference 4(1H)-quinolone 1c. In contrast, EC₅₀ values for ester 3 and carbonate 4 were determined to be comparable to the EC₅₀ values of 1c. These results suggest that the ester and carbonate prodrugs are spontaneously hydrolyzed into the active parent molecule lc during the multiday incubation conditions, whereas compounds 2a and 2b are most likely inactive due to the high stability of the carbamate group. Despite the previous reported increased oral bioavailability utilizing an ethyl carbonate prodrug of 4(1H)-quinolone 1a,³⁴ carbamate (2a, <1.0 μM and 2b, 2.9 μM, 2b), ester (3, 7.9 μM) and carbonate (4, 1.2 μM) linked promoieties do not noticeably improve the aqueous solubility of reference 1c (8.7 μM).

To optimize the compromise between carbonate prodrug's stability and the release rate of corresponding parent 4(1H)-quinolone, especially electron withdrawing 4(1H)-quinolones, a methylene bridge was introduced between the carbonate group and the 4-quinolinol's oxygen, leading to an alkoxycarbonyloxymethyl (AOCOM) ether prodnig.³⁹ Using the AOCOM approach, the promoiety's carbonyl group is electronically insulated from the 4(1H)-quinolone's core, which aids to the stability of the prodrug. It was previously shown that linking of an ethoxycarbonyloxymethyl promoiety onto an aromatic hydroxy group of parent drugs lead to prodrugs that are stable to chemical hydrolysis at pH 1 (t_{1/2}≥50 h) and undergo slow hydrolysis (t_{1/2}≥8 h) at pH 6.0-8.5.⁴⁰

Further control over the release rate was gained by integrating an ionizable amino group into a particular position of the AOCOM residue, in which the amino group's nucleophilicity can be utilized in a pH-dependent mechanism to release the parent compound from the corresponding prodrug.⁴¹ This amino group was also introduced to significantly improve the limited aqueous solubility of carbonate prodrugs. Protonation of the amino group at a low pH (e.g. upper GIT pH) should lead to a stable prodrug with a substantially enhanced aqueous solubility, vhereas an increase in pH should gradually deprotonate the amino group. This will steadily increase the nitrogen's nucleophilic character and accelerate the release of the parent compound via an intramolecular cyclization reaction (FIG. 3a). The introduced methylene bridge between the parent compound and the carbonate group ensures the applicability of this prodrug approach to any 4(1H)-quinolone without influencing the electrophilicity of the promoiety's carbonate carbon, which is important for the release process. As a result of this pH-triggered release mechanism, the amino-based AOCOM prodrug approach is independent of any enzyme activity avoiding inter- and intra-species variabilities, which potentially complicate clinical studies or future therapeutic applications. During the release of the parent 4(1H)-quinolone, formaldehyde as well as a cyclic carbamate ring, whose ring size depends on the length of the promoiety, are formed as side products. Importantly, neither the lactam nor formaldehyde should lead to toxicity at the intended dose levels.

Analogous to known AOCOM ether prodrugs, AOCOM iodides were used to prepare the desired prodrugs.³⁹ Thus, the synthesis route to the prodrugs commences with conversion of Boc-protected amino alcohols into corresponding Boc-protected aminoalkyloxycarbonyloxymethyl (amino AOCOM) iodides (FIG. 4a). Reaction of 4-quinolinols 1c′ and with the 1d′ with the Boc-protected amino AOCOM iodides, in presence of caesium carbonate, and subsequent deprotection leads to water-soluble HCl salts 6a-d (FIG. 4b). 4(1H)-Quinolone 1d was chosen as parent compound as it was shown to possess enhanced microsomal stability over 1c due to the CF₃-substituted phenyl moiety in 3-position. The assessment of the aqueous solubility at pH 7.4 was challenging due to the limited stability of the prodrug moiety at neutral pH and was not performed. Aqueous solubility at pH 2.5 was therefore determined using a precedented UV-based assay.²⁸ For amino AOCOM ether prodrugs 6a and 6b, an aqueous solubility of over 100 μM at acidic pH was determined. In addition, solid material was rapidly dissolved in 0.5% aqueous HEC solution and visually inspected after 60 seconds for material dissolution. The observed solubility of >20 mM for amino AOCOM ether prodrugs of more soluble 1c and >5 mM for amino AOCOM ether prodrugs of less soluble 1d illustrate the significant increase in solubility and dissolution rate enabled by the use of amino AOCOM ethers.

Prodrugs 6c and 6d show suitable aqueous stability-release profiles (FIGS. 3b and 3c). Compound stability was assessed using quantitative HPLC in buffers at different pHs (2, 4, 7), in a simulated gastric fluid (SGF) and in a simulated intestinal fluid (SIF). Generally, in all tested solutions, 6d appeared to be more stable than 6c. Compound 6c rapidly released parent compound 1d at pH 7.0 and pH 4.0 (>90% parent compound released in 1 h at pH 7.0 or >25% parent compound released in 10 h at pH 4.0). In comparison, prodrug 6d was stable at low pH values and decomposed slowly, releasing parent compound 1d, at pH 7.0 (>55% parent compound released in 5 h at pH 7.0). These results show that the parent compound release can be adjusted in terms of pH and rate. Of the two promoieties, the one containing a three methylene spacer between the carbonate oxygen and the methylamino group possesses the most promising pH-stability profile. It should release the parent compound slowly in the intestine so that it can be absorbed continuously. In contrast, the prodrug with the two methylene spacer could precipitate in the upper GIT because it would be released too early and too quickly.

Nevertheless, all amino AOCOM ether prodrugs of 1c and 1d (6a, 6c with a two and 6b, 6d with a three methylene groups-containing promoiety) were selected to undergo in vivo efficacy testing using a modified Thompson test model, which was previously reported (see Table 1).^{29, 32, 33} However, parent compounds 1c and 1d and prodrugs 6a-6d were administered in 0.5% aqueous HEC instead of neat PEG 400. Parent compounds displayed poor activity with 29% or lower suppression of parasitemia on day 6 post infection (PI). In contrast, at both doses (10 mg/kg and 50 mg/kg), the four amino AOCOM ether prodrugs 6a-6d proved more efficacious in vivo than parent compounds 1c and 1d, demonstrating the viability of amino AOCOM ether prodrugs in an in vivo setting (see Table 1). Prodrugs 6c and 6d, which contain a p-(trifluoromethyl) phenyl group in the parent 4(1H)-quinolone's 3-position, are slightly more active than prodrugs 6a or 6b, which are substituted with a o-methyl phenyl moiety in 3-position. Compound 6d, which contains the prodrug moiety that includes a three methylene spacer between the carbonate and the methylamino group, was able to suppress parasitemia by 82% at a 10 mg/kg dose and 96% at a 50 mg/kg dose. The dose linearity for prodrug 6d was proven in another series of tests “Dose Linearity of Amino AOCOM Ether Prodrug”). Nevertheless, 6d do not significantly extend the life span of treated animals relative to untreated controls. These findings are not unexpected as parent 4(1H)-quinolone 1d was shown to exhibit rapid clearance in vivo.³²

TABLE 1
In vivo efficacy of 1c, 1d and their amino AOCOM ether prodrugs 6a-d.^{a}

| | 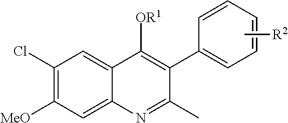 | | 10 mg/kg (PO)^{b} | | 50 mg/kg(PO)^{b} | |
|---|---|---|---|---|---|---|
| | | | Suppression | Day of | Suppression | Day of death |
| No | R¹ | R² | [%] day 6 PI^{c} | death (avg) | [%] day 6 PI^{c} | (avg) |
| 1c′ | —H | o-CH₃ | 29 | 13 | 28 | 13 |
| 6a | 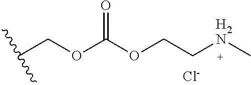 | o-CH₃ | 71 | 16 | 46 | 13 |
| 6b | 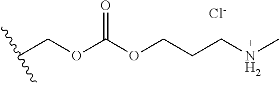 | o-CH₃ | 52 | 13 | 72 | 13 |
| 1d′ | —H | o-CH₃ | 11 | 14 | 10 | 13 |
| 6c | 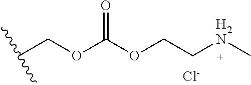 | m-CF₃ | 75 | 13 | 96 | 16 |
| 6d | 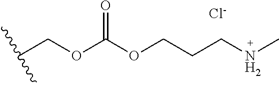 | m-CF₃ | 82 | 13 | 96 | 14 |
| untreated | — | — | 0 | 13 | — | — |
| ADQ | — | — | >99 | 21 | n.d. | n.d. |

^{a}Mice were infected with 1-10⁸ P. berghei-GFP parasites and then orally treated once a day on days 3-5 PI with test compound in a 0.5% aqueous HEC solution. Parent compounds 1c and 1d were administered as controls using the same protocol.
^{b}Oral administration as three daily doses (formulated in 0.5% aqueous HEC) on days 3-5 PI.
^{c}Percent supression as compared to untreated control animals.

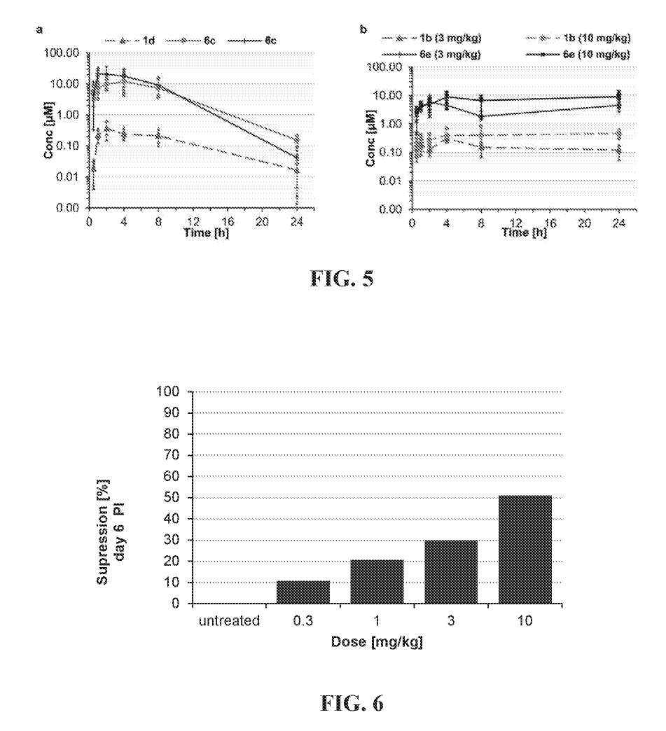

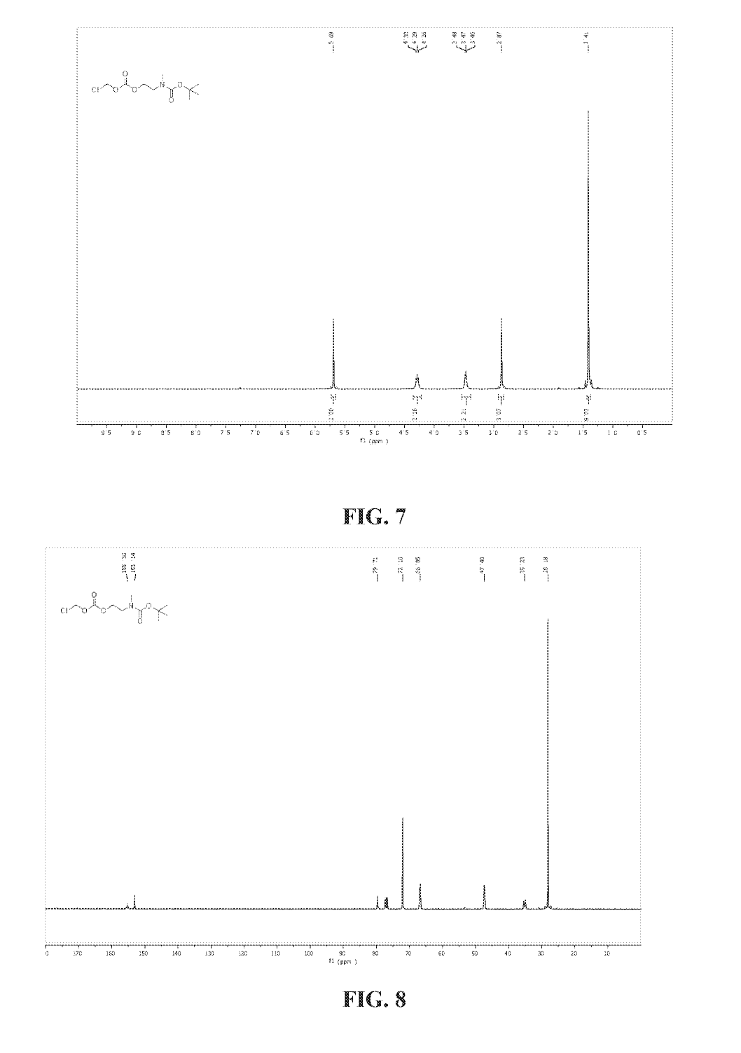

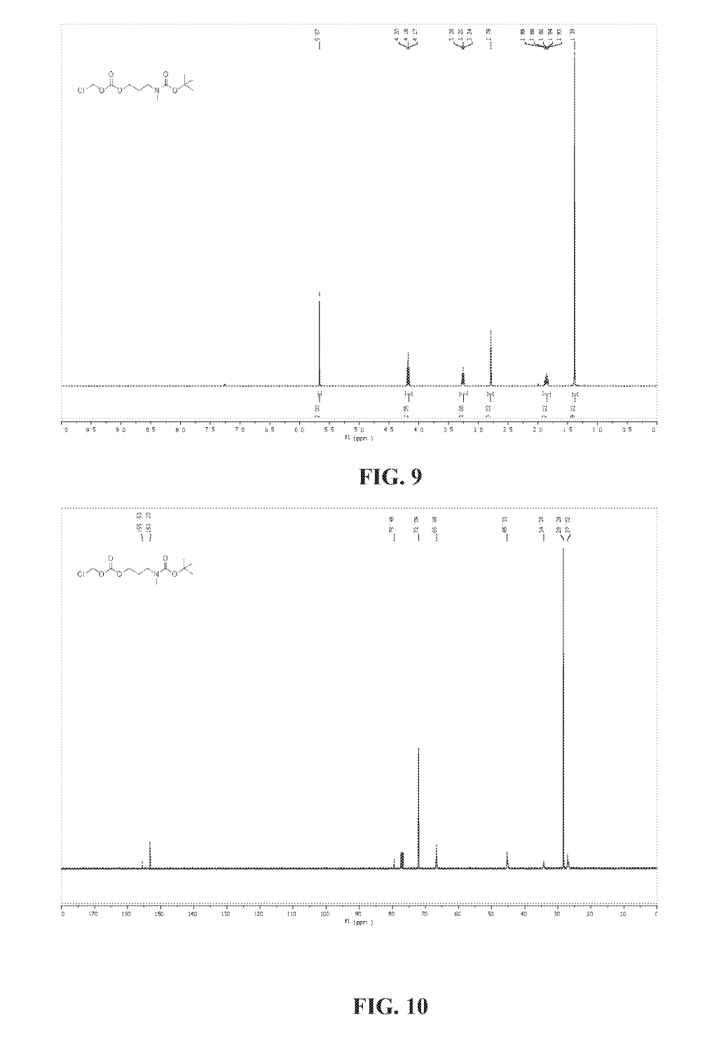

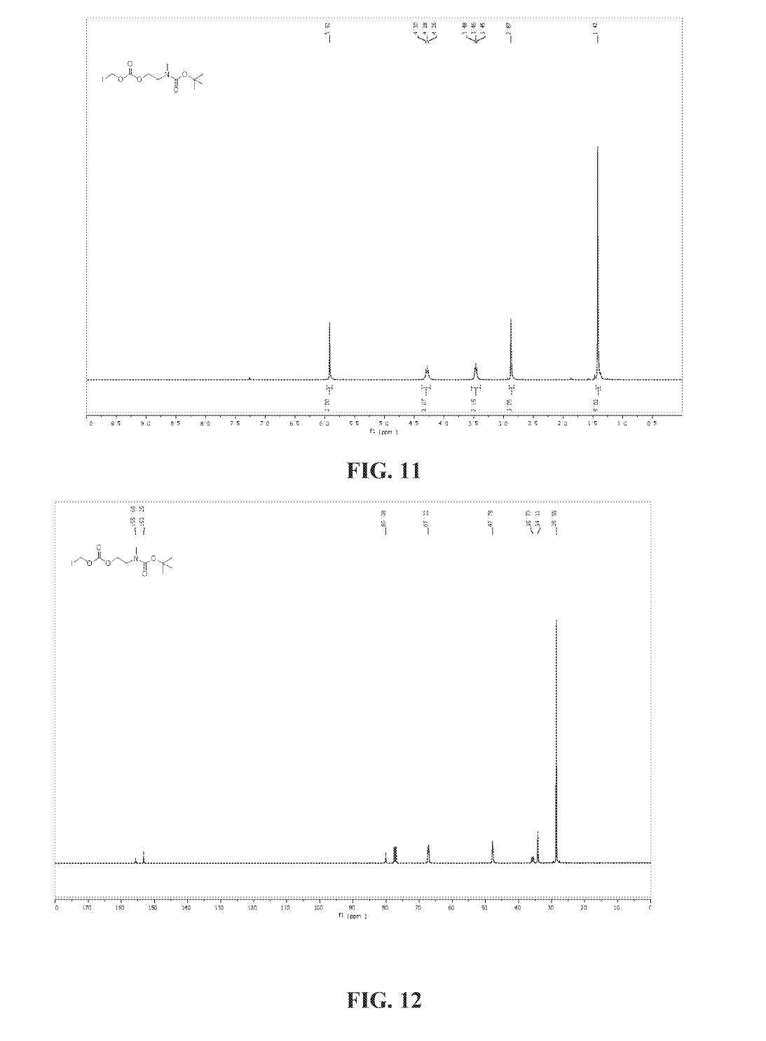

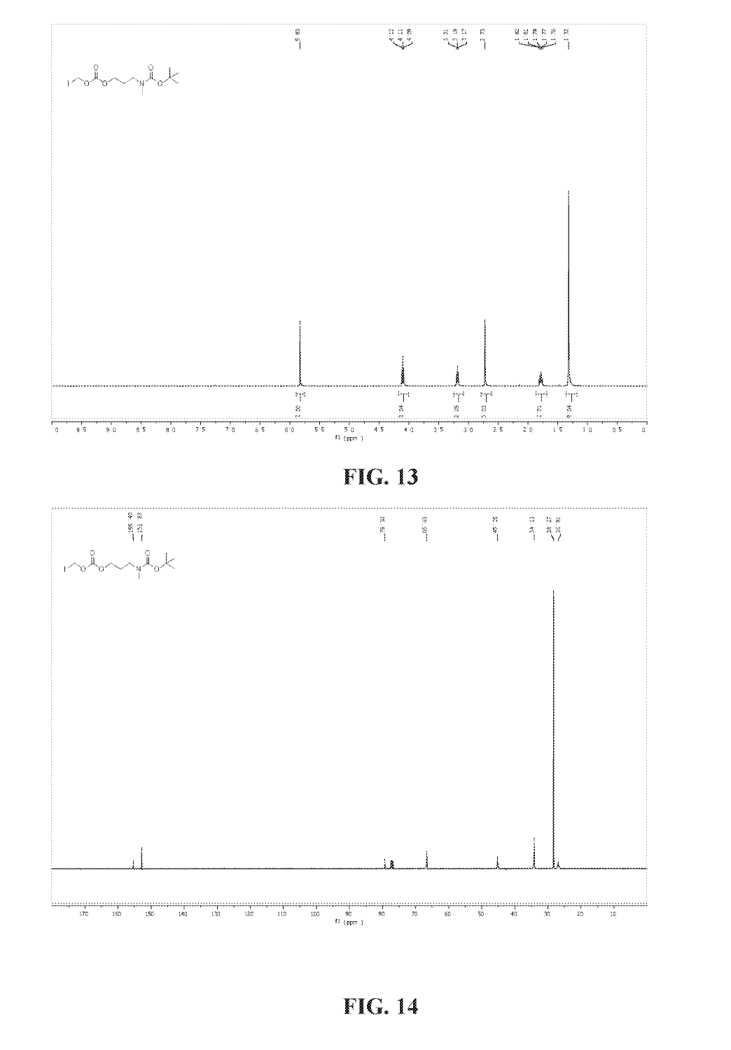

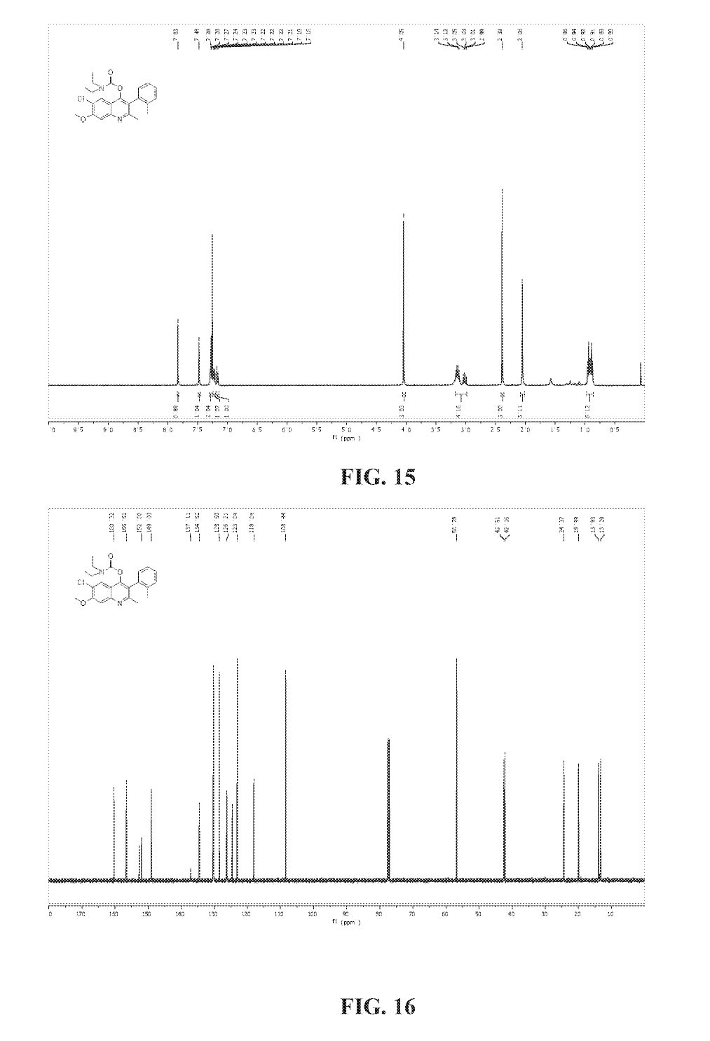

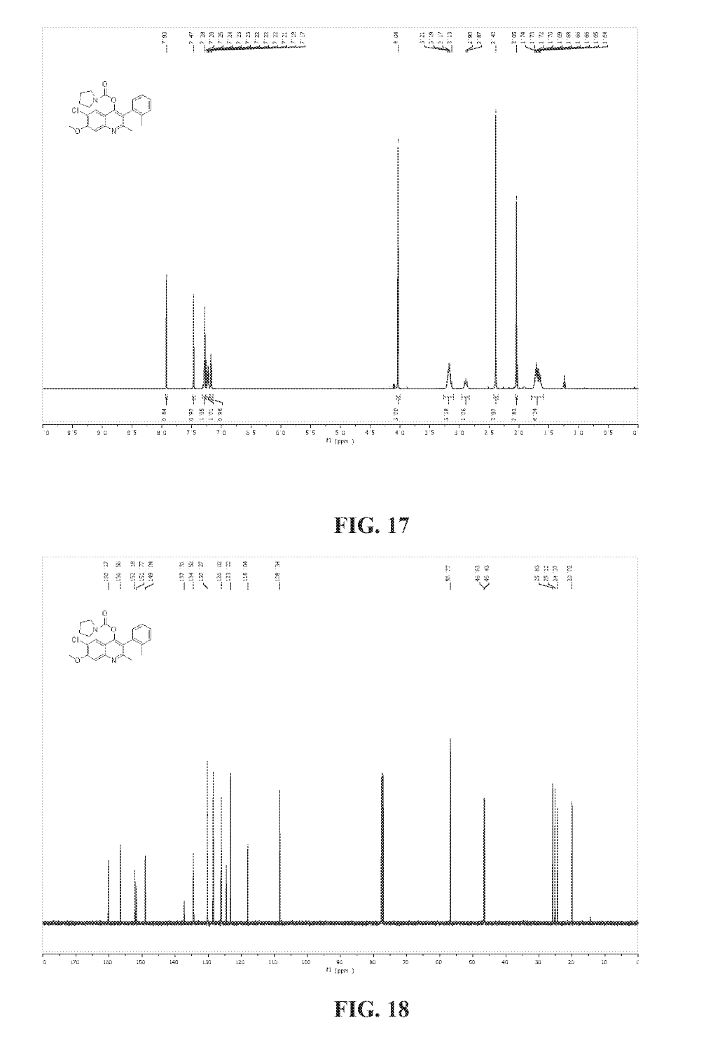

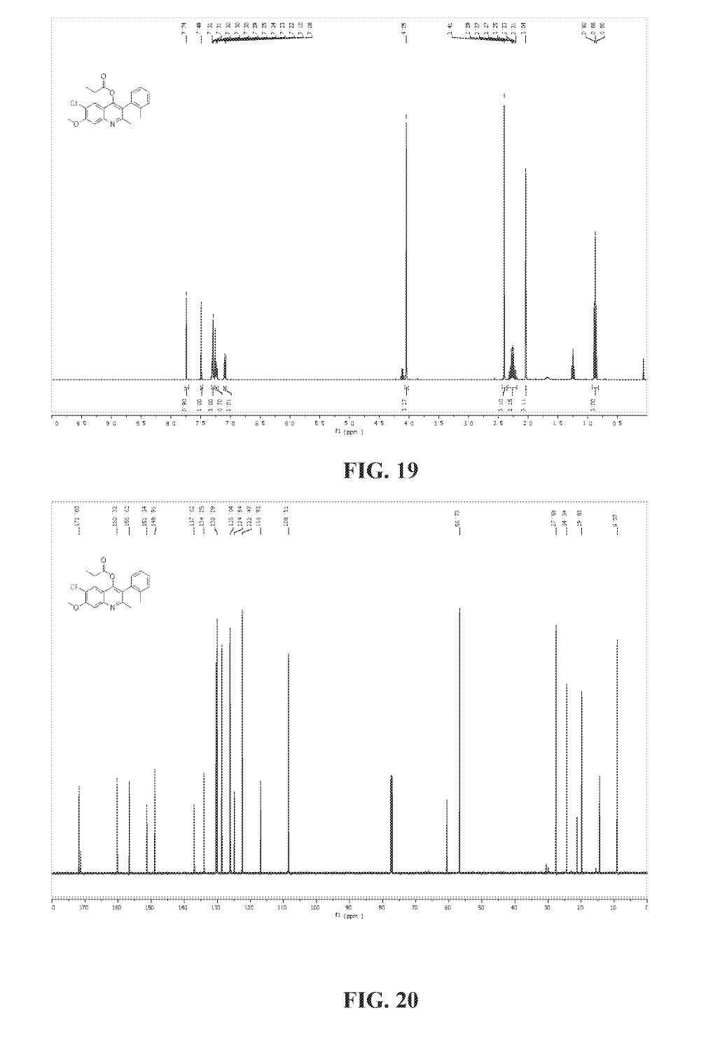

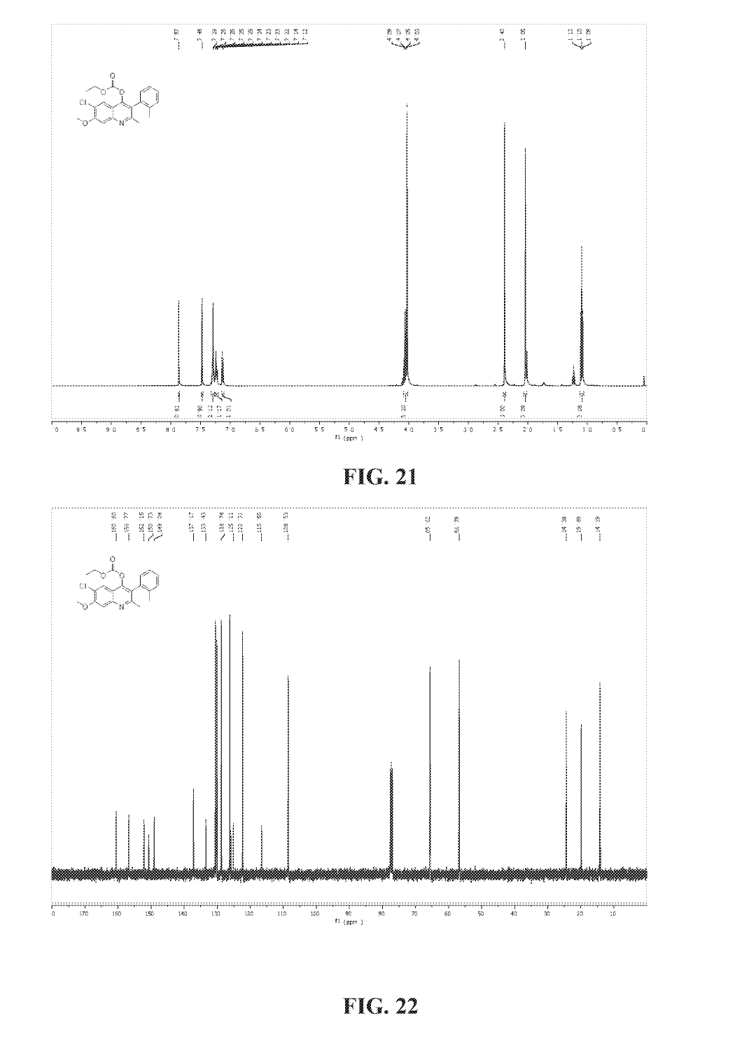

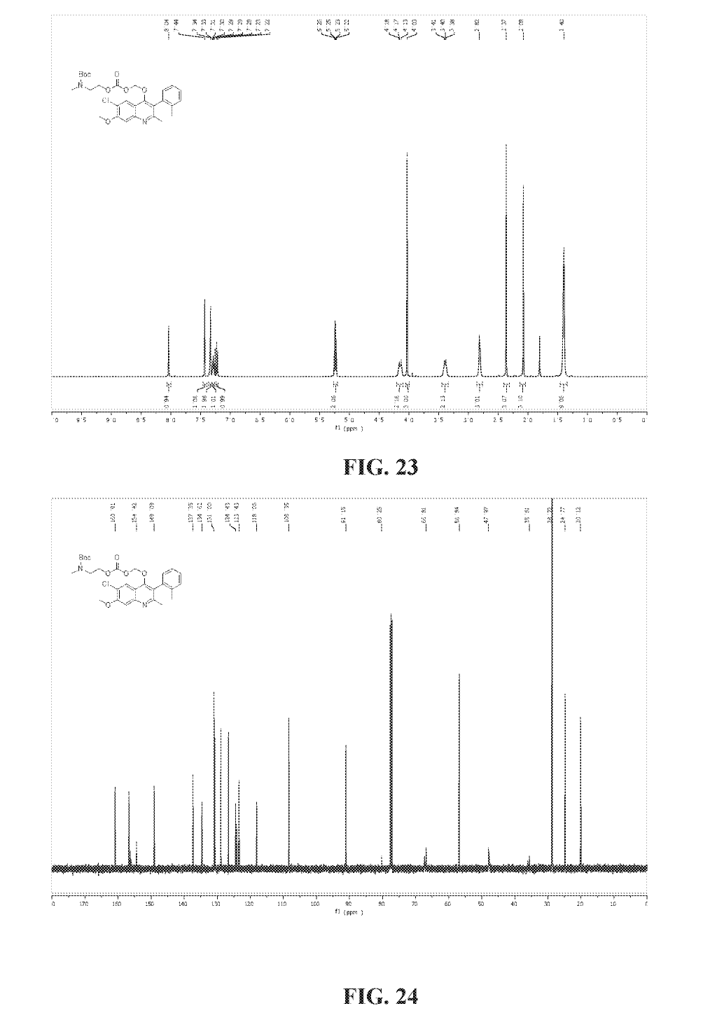

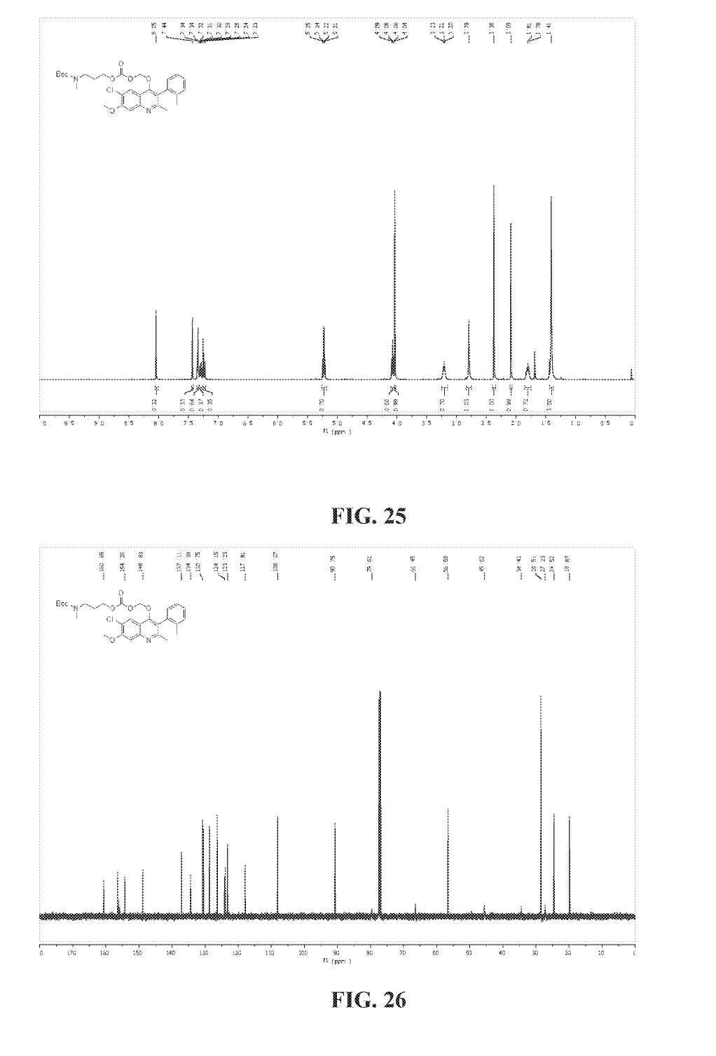

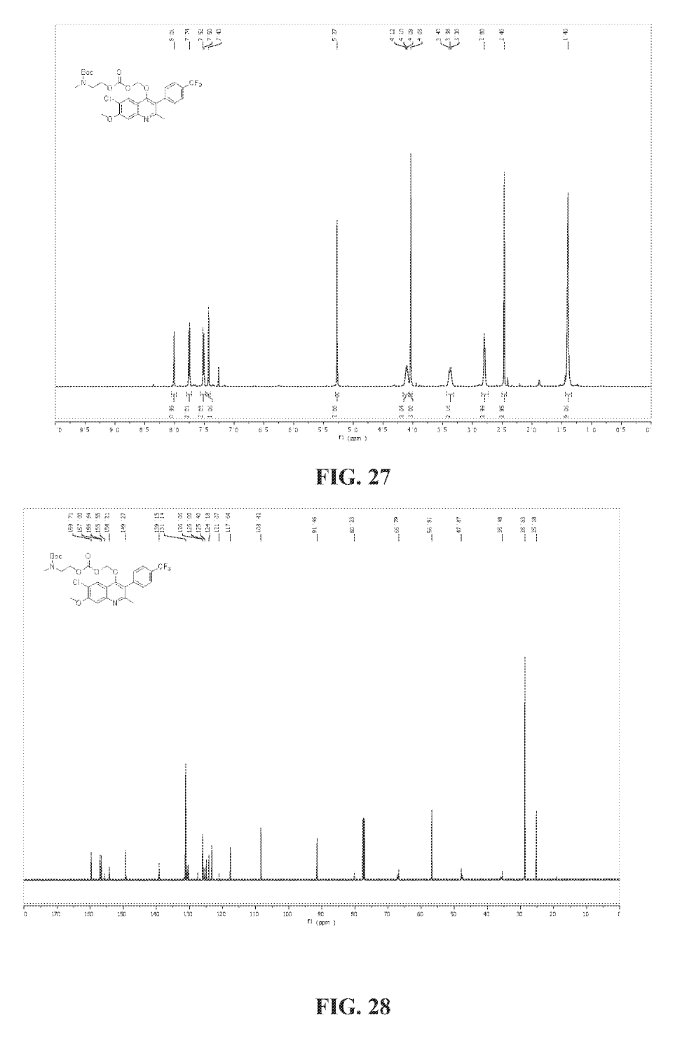

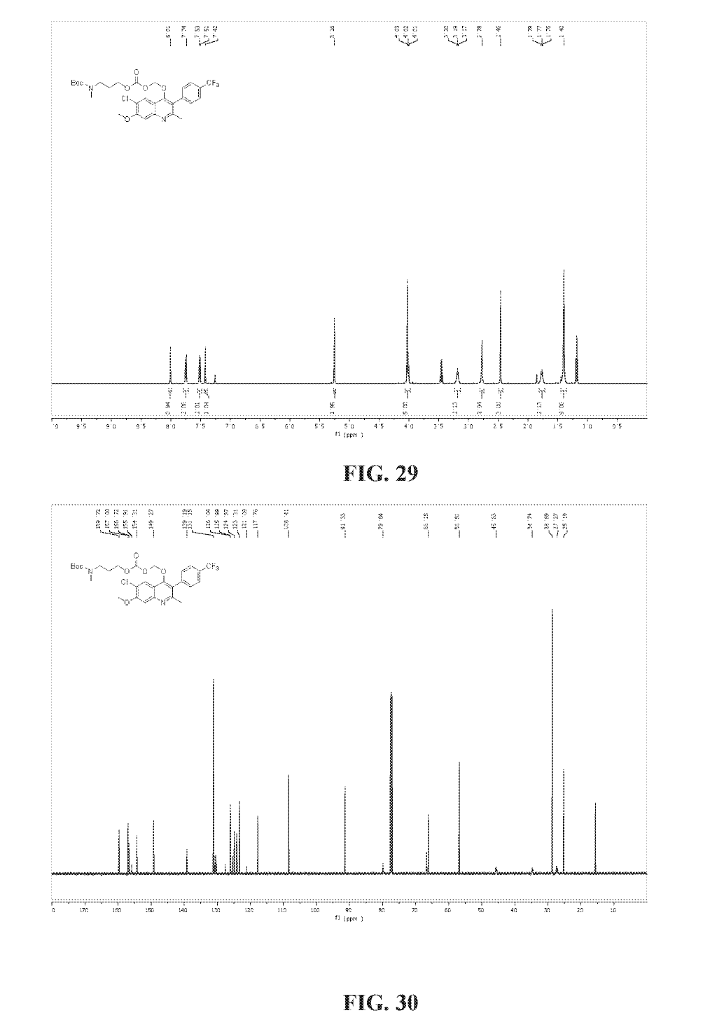

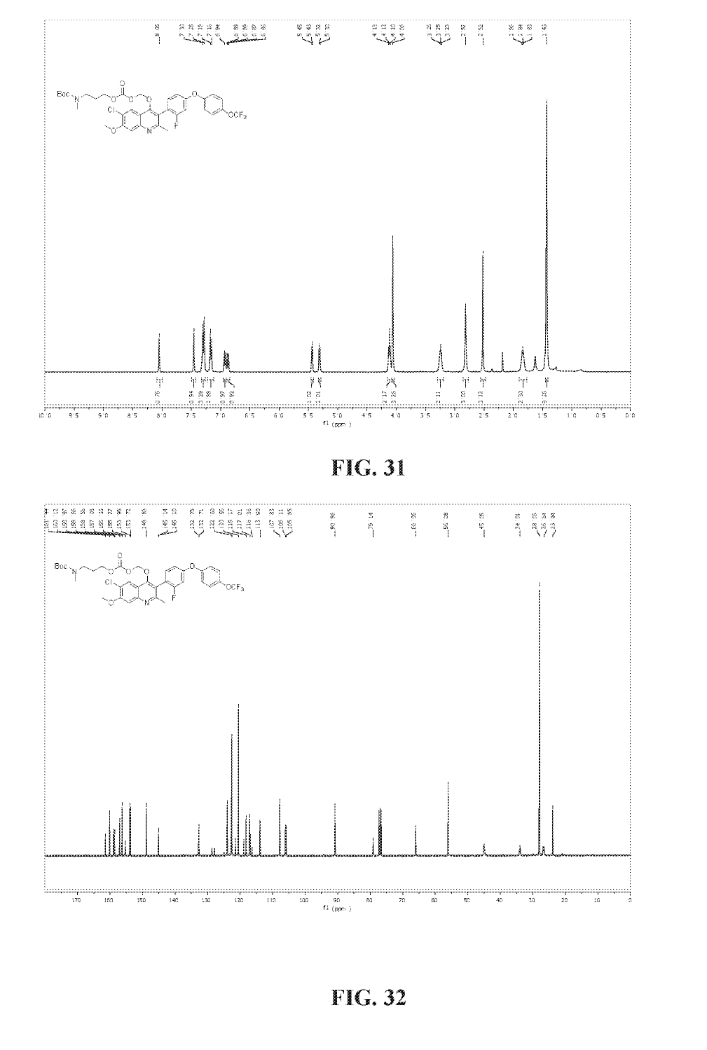

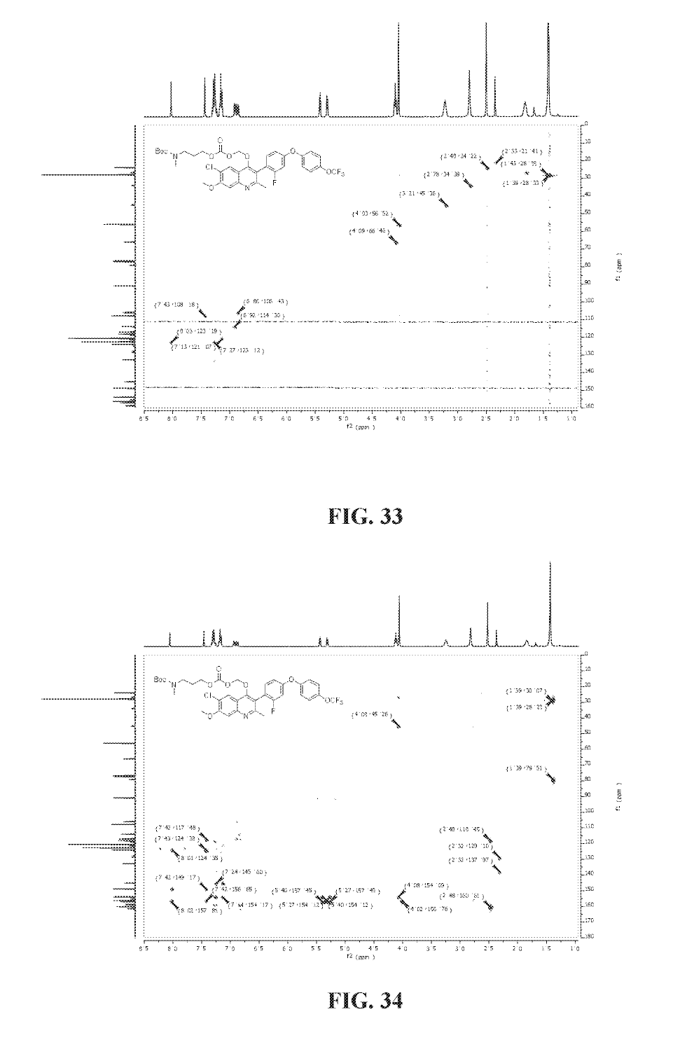

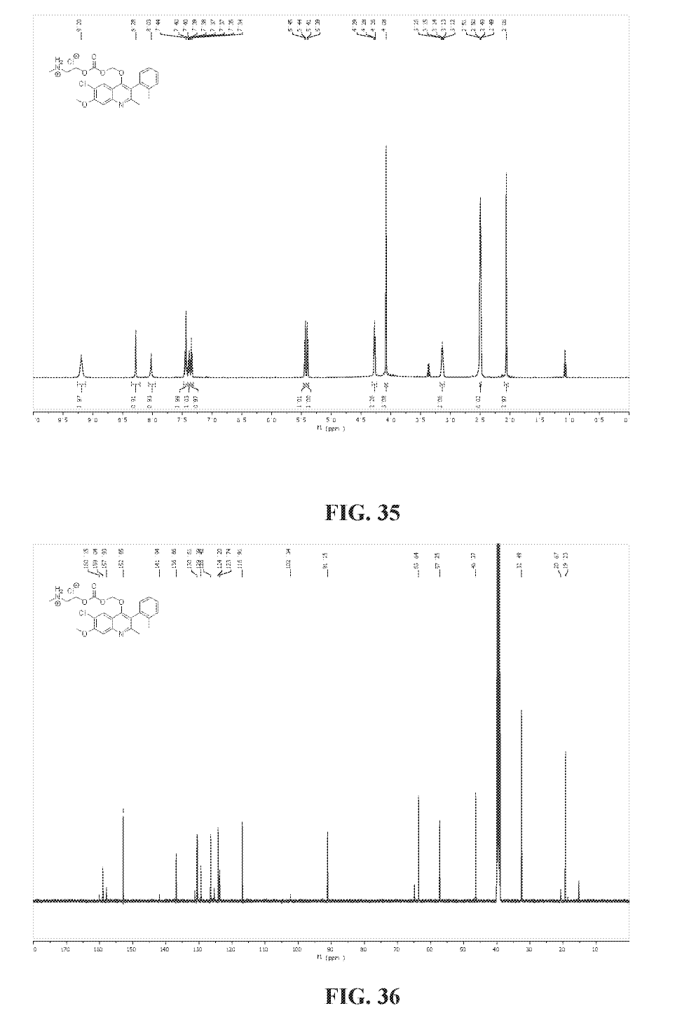

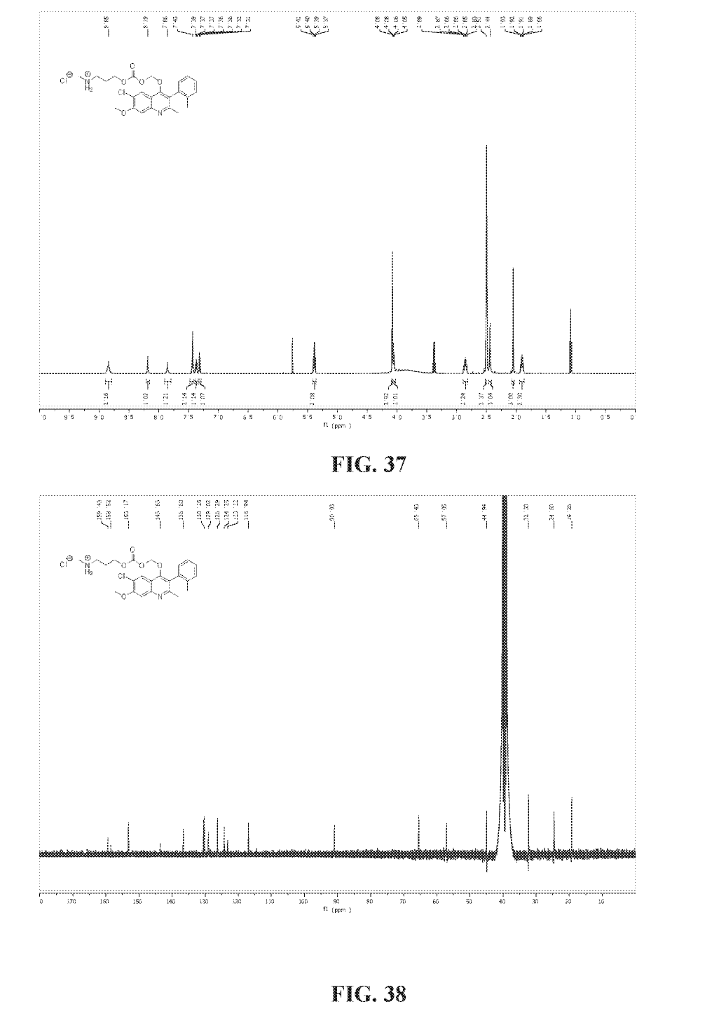

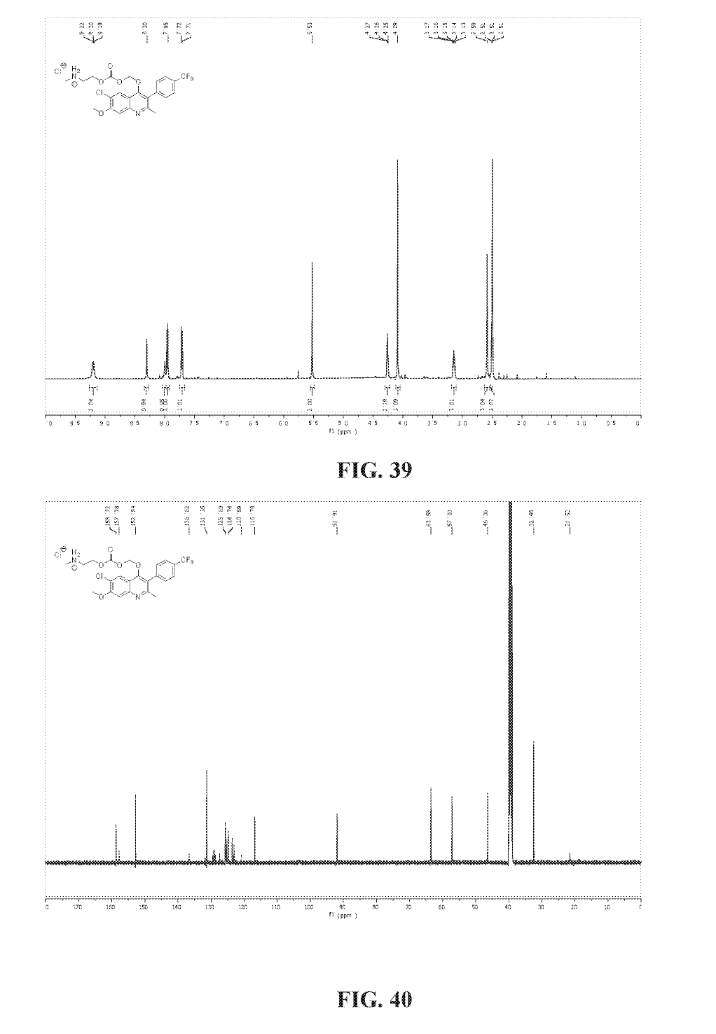

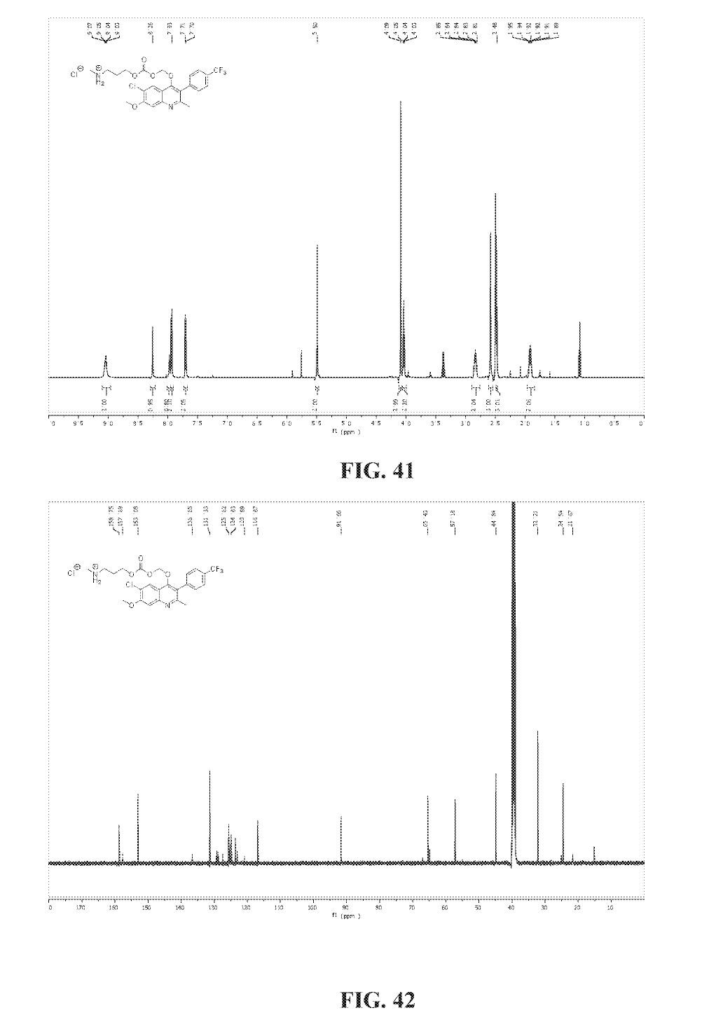

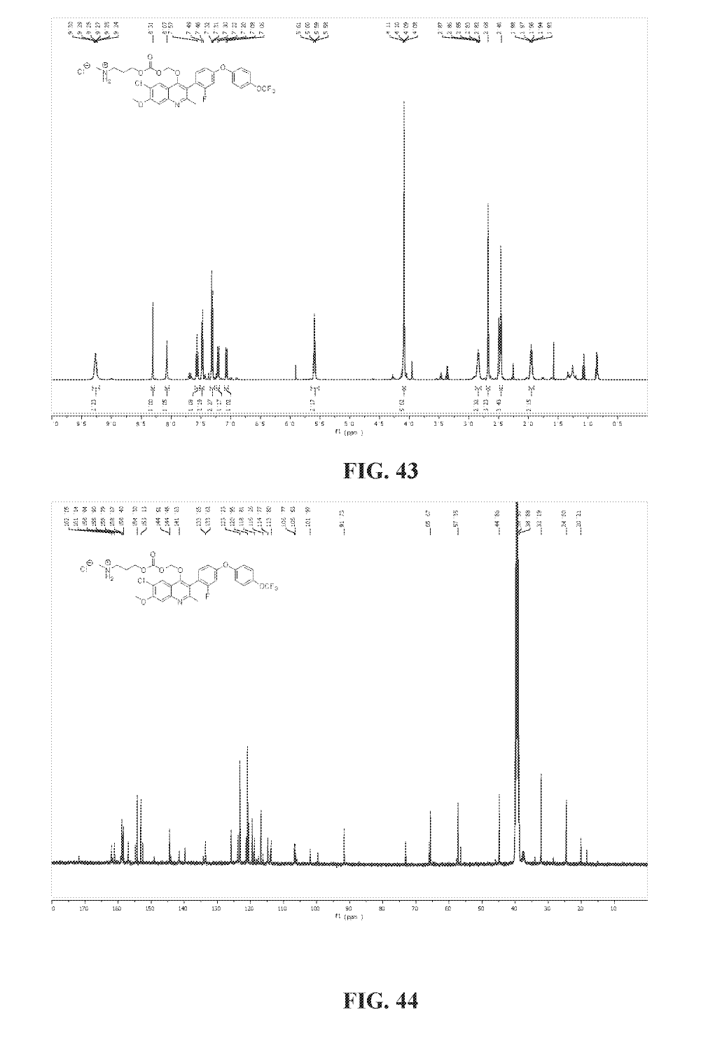

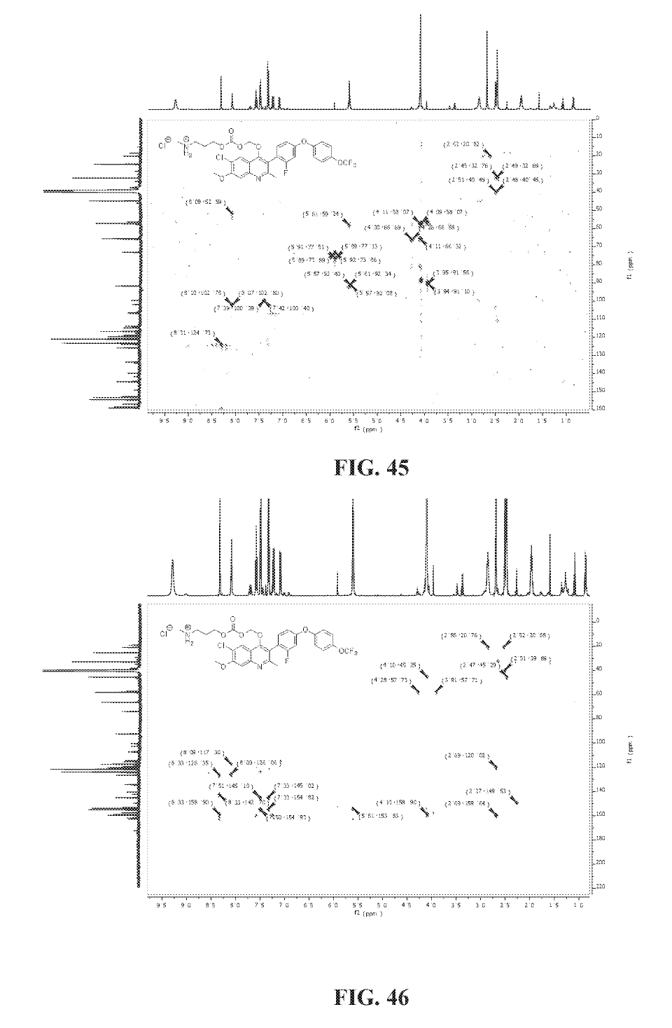

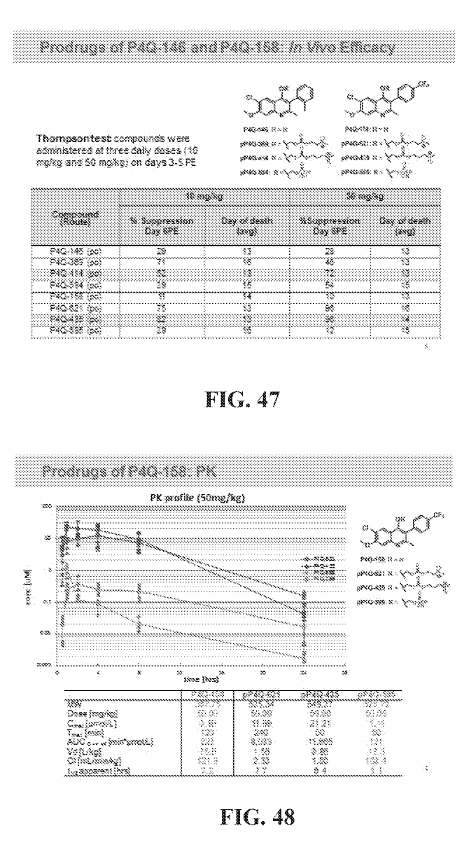

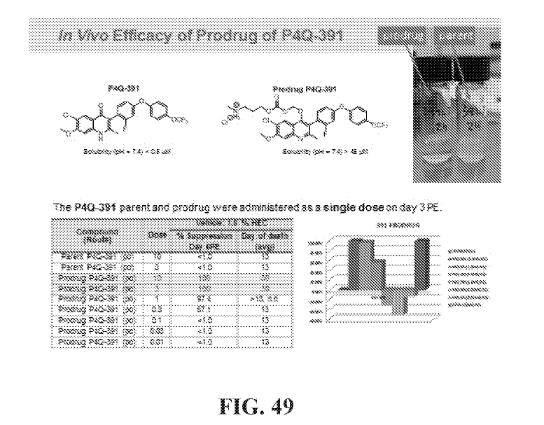

Phartnacokinetic studies with the two most potent prodrugs 6c and 6d as well as their parent compound 1d were conducted to profile compound plasma exposure after single oral administration at a dose of 50 mg/kg in 0.5% aqueous HEC formulation (FIG. 5a). 4(1H)-quinolone 1d was slowly absorbed, reaching a maximum plasma concentration of 365 nM, 2 hours after oral dosing of 1d. Importantly, the concentration of 1d remained above the lower limit of quantification (LLQ) of 1 nM over the course of the entire profiling experiment. The maximum plasma concentration of 1d significantly increased for prodrugs 6c and 6d to 12 μM and 21 μM, whereas AUC of 1d improved by 36-fold for 6c and 52-fold for 6d (see Table 2). As previously observed with orally dosed 1d,³² parent compound 1d was rapidly cleared in less than 24 hours for both prodrugs 6c and 6d, which led to the observed increase in suppression without significantly extending survival.

TABLE 2
PK parameters of parent 1d, 1b and their amino AOCOM
ether prodrugs 6c, 6d, 6e after oral administration of test
compound in a 0.5% aqueous HEC solution.

| | Id | 6c | 6d | 1b | | 6e | |
|---|---|---|---|---|---|---|---|
| MW | 367.7 | 535.3 | 549.4 | 493.8 | | 675.4 | |
| dose^{a} [mg/kg] | 50.0 | 50.0 | 50.0 | 3.0 | 100 | 3.0 | 10.0 |
| Cₘₐₓ [μmol/L] | 0.36 | 12.0 | 21.2 | 0.31 | 0.46 | 5.7 | 9.1 |
| tₘₐₓ [min] | 120 | 240 | 60 | 240 | 480 | 120 | 240 |
| AUC_{0→24} | 222 | 8031 | 11664 | 228 | 580 | 4818 | 10269 |
| [min · μmol/L] | | | | | | | |
| V_{d} [L/kg] | 377.7 | 7.8 | 4.3 | 19.6 | 44 | 0.8 | 1.6 |
| CL [mL · kg/min] | 609.7 | 11.6 | 7.8 | 26.6 | 34.9 | 0.9 | 1.4 |
| t_{1/2 apparent} [h] | 7.15 | 7.7 | 6.4 | 8.5 | 14.5 | 9.7 | 13.1 |

Antimalarial compounds with potent activity and a long in vivo half-life have potential to be curative single-dose agents. It was hypothesized that installation of an amino AOCOM ether promoiety onto 1b would deliver a curative single-dose agent, because, in comparison to 1d, 4(1H)-quinolone 1b was previously reported to display low in vivo clearance following oral administration in addition to excellent^{32, 33} Of the two prodrug moieties, the one with a three methylene group spacer between the carbonate and the methylamino group was considered to be the most promising, due to the optimized pH-stability (FIG. 3c), the improved in vivo efficacy (Table 1), and the enhanced pharmacokinetic profile (FIG. 5a) when comparing to parent compound and other prodrugs. Prodrug 6e was synthesized in an analogous manner to 6a-d (FIG. 4a),

Plasma exposure of prodrug 6e and parent compound 1b was determined following a single oral administration at 10 mg/kg and 3 mg/kg doses in 0.5% aqueous HEC (FIG. 5b). Overall, prodrug 6e performed better than parent compound 1b at both doses, increasing Cₘₐₓ and AUC of 1b approximately 20-fold. For example, at a 3 mg/kg dose, a Cₘₐₓ of 5.74 μM was determined for 6e, whereas a Cₘₐₓ of a 0.31 μM was determined for 1b. Importantly, 18-fold improvements of Cₘₐₓ and 21-fold improvements in AUC were achieved without the use of any advanced formulation techniques. This constitutes a major advancement when compared to the carbonate prodrug approach developed by Riscoe and co-workers in which the plasma exposure of parent 1a only enhanced by ˜3-fold (PEG 400 formulation, 3.5 mg/kg PO dose of prodrug).³⁴ In a manner identical to previously reported pharmacokinetic studies with parent compound 1b in neat PEG 400,³³ prodrug 6e exhibited low clearance with a long half-life of 10 hours or longer. This in conjunction with the increased Cₘₐₓ and AUC values implied prodrug 6e to be sufficiently bioavailable to produce single-dose cures at a dose as low as 3 mg/kg.

P. berghei-infected mice were treated orally with a single dose of parent compound 1b or prodrug 6e on day 3 PI. Prodrug 6e was administered in 0.5% aqueous HEC formulation at doses ranging from 0.01 mg/kg to 10.0 mg/kg whereas, for comparative reasons, parent 4(1H)-quinolone 1b was dosed at 10 mg/kg. Remarkably, prodrug 6e was curative at both 3 mg/kg and 10 mg/kg, whereas parent compound 1b was completely inactive when administered as a single dose of 10 mg/kg using the same vehicle (Table 3). To the best of our knowledge, a single oral dose of 3 mg/kg is the lowest dose among all antimalarials that are currently in clinical trials (the closest candidate trioxolane OZ439, which is administered in combination with ferroquine, cures P. berghei-infected mice with a single oral dose of 20 mg/kg) producing curative activity. Furthermore, prodrug 6e at a 1 mg/kg oral dose showed 97% suppression of parasitemia on day 6 PI extending the average day of death for all 5 mice beyond day 13 (the average suppression of parasitemia on day 13 was 90%).

TABLE 3
In vivo efficacy of 1b and its amino AOCOM ether prodrug 6e.^{a}

| No | 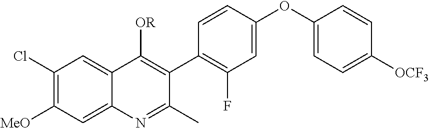 | Dose (PO) [mg/kg] | Suppression [%] day 6 PI^{b} | Day of death (avg) |
|---|---|---|---|---|
| 1b′ | —H | 10^{c} | <1 | 13 |
| 6e | 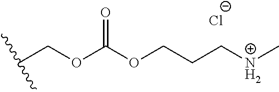 [SMILES: *COC(=O)OCCC[NH2+]C ; [Cl-]] | 10^{c} 3^{c} 1^{c} 0.3^{c} 0.1^{c} | >99 >99 97 57 <1 | curative curative 21 13 13 |
| | | 0.03^{c} | <1 | 13 |
| | | 0.01^{c} | <1 | 13 |
| untreated | — | — | 0 | 13 |
| ADQ | — | 10^{d} | >99 | 21 |

^{a}Mice were infected with 1-10⁶ P. berghei-GFP parasites and then orally treated with a single dose on day 3 PI with test compound in a 0.5% aqueous HEC solution. Parent compound 1e was administered as control using the same protocol.
^{b}Percent suppression as compared to untreated control animals.
^{c}Oral administration as single dose (Formulated in 0.5% aqueous HEC) on day 3 PI.
^{d}Oral administration as three daily doses (formulated in 0.5% aqueous HEC) on days 3-5 PI.

To demonstrate the versatility of the developed prodrug approach, the amino AOCOM prodrug moiety was successfully installed in ICI56,780, another poorly soluble 4(1H)-quinolone with antimalarial activity (see ‘Amino AOCOM Prodrug of ICI56,780’). As expected, the prodrug of ICI56,780 noticeably improved exposure and in vivo efficacy in comparison to parent compound ICI56,780.

All reagents and solvents were purchased from commercial sources and used without further purification unless noted otherwise. All reactions were carried out under an argon atmosphere using flame-dried glassware and standard. Schlenk techniques unless indicated otherwise. Prior to use of solvents in reactions, they were purified by passing the degassed solvents through a column of activated alumina and transferred by an oven-dried syringe or cannula. The identity of all title compounds was verified via ¹H NMR, ¹³C NMR, and HRMS. The chemical purity of the title compounds was determined by LC/MS using the following instrumentation and the following analytical conditions: an Agilent 1100 series LC/MSD equipped with a Phenomenex Kinetex reversed phase column (50 mm×4.6 mm, 2.6 μm, C18, 100 Å); method: 10% (v/v) of acetonitrile (+0.1% FA) in 90% (v/v) of H₂O (0.1% FA), ramped to 100% acetonitrile (0.1% FA) over 5.5 min, and holding at 100% acetonitrile (0.1% FA) for 1 min with a flow rate of 1.3 mL/min; UV detector, 254 nm. The purity of each compound was ≥95% in this analysis. ¹H NMR spectra were recorded at ambient temperature on a 400, 500 or 600 MHz Varian NMR spectrometer in the solvent indicated with the signal of the residual solvent (CHCl₃ δ 7.26 ppm; DMSO-d₆ δ 2.50 ppm)⁴² as internal standard. ¹³C NMR spectra were recorded with ¹H decoupled observation at ambient temperature on a Varian NMR spectrometer operating at 100, 125 or 150 MHz in the solvent indicated with the signal of the residual solvent (CHCl₃ δ 77.1 ppm; DMSO-d₆ δ 39.5 ppm)⁴² as internal standard. ¹⁹F NMR. spectra were recorded on a Varian NMR spectrometer operating at 376 MHz with 3-nitrofluorobenzene (−112.0 ppm) as added standard. Data for ¹H NMR are reported as follows: chemical shift (ppm), multiplicity (s=singlet, br=broad, d=doublet, t=triplet, q=quartet, sept=septet, m=multiplet), coupling constant (Hz), and integration. For the ¹³C and ¹⁹F NMR data chemical shifts (δ ppm) and multiplicities (if not a singlet) are given. NMR data was processed using MestReNova Software ver. 8.1. ¹³C signals arising from carbons next to the Boc-protected or protonated nitrogen are often broad or even splitted into two signals. In those cases, the central of the signal is given. The ¹H and ¹³C signals of compound 5e and 6e were assigned by using ¹H/¹H COSY, ¹H/¹³C HSQC and ¹H/¹³C HMBC. The O-alkylation instead of a possible N-alkylation was verified by that. By comparison of the spectra this verification is transferrable to all other prodrug compounds. High resolution mass spectra (HRMS) were performed on an Agi lent LC/MS Q-TOF system 6540 UHD. Compounds were eluted using a gradient elution of 70/30 to 50/50 A/B over 30 minutes at a flow rate of 5.0 mL/min, where solvent A was 0.1% TFA in water and solvent B was 0.1% TFA in acetonitrile. Analytical thin layer chromatography (TLC) was performed on silica gel 60 F254 pre-coated plates (0.25 mm) from EMD Chemical Inc. and components were visualized by ultraviolet light (254 nm). Silica gel 60 from EMD Chemical Inc. with 230-400 (particle size 40-63 μm) mesh was used for all flash column chromatography. Purified compounds were further dried under high vacuum to e ove residual solvent. Yields given in the Sipplementary information refer to purified compounds. All 4(1H)-quinolones (6-chloro-3-(2-fluoro-4-(4-(tfifluoromethoxy)phenoxy)phenyl)-7-methoxy-2-methyl -4(1H)-quinolone (1b), 6-chloro-7-methoxy-2-methyl-3-(o-tolyl)-4(1H)-quinolone (1c), and 6-chloro-7-methoxy-2-methyl-3 -(4-(trifluoromethyl)phenyl)-4(1H)-quinolone (1d)) were prepared as previously described via a Conrad-Limpach cyclization of 2-aryl substituted ethyl acetoacetate with 3-chloro-4-methoxy aniline.²⁰ The Boc-protected aminoalcohols 2-((tert-butoxycarbonyl)(methyl)amino)ethanol⁴³ and 3-((tert-butoxycarbonyl)(methyl)amino)propanolwere prepared as described in the literature.

### General Procedure A (GP A) for the Formation of Chloromethyl Carbonates (S1)

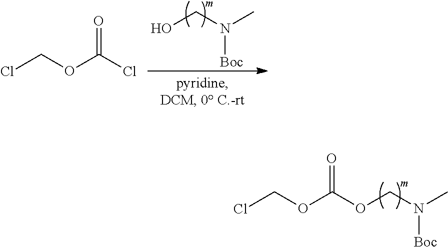

A round-bottom flask was charged with a solution of the respective alcohol in DCM (2.5 mL/mmol acohol). The stirred solution was cooled to 0° C. and pyridine was added. Then, chloromethyl chloroformate, dissolved in DCM (1.0 mL per mmol nucleophile), was added dropwise. The mixture was allowed to stir at room temperature for 10 h. The reaction was quenched with 3M HCl_{(aq)} (3.0 mL/mmol nucleophile) and extracted with DCM (3×2.0 mL/mmol nucleophile). The combined organic layers were washed with coned NaHCO_{3(aq)} (4.0 mL/mmol nucleophile) and brine (4.0 mL/mmol nucleophile), dried over Na₂SO₄ and concentrated under reduced pressure. The pure product was obtained without any further purification.

Chloromethyl 2-((tert-butoxycarbonyl)(methyl)amino)ethyl carbonate (S1a)

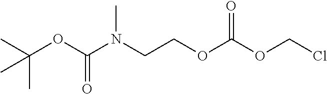 [SMILES: CN(CCOC(=O)OCCl)C(=O)OC(C)(C)C]

According to GP A, chloromethyl carbonate S1a was prepared reacting 2-((tert-butoxycarbonyl)(methyl)amino)ethanol (3.15 g, 18.0 mmol), pyridine (1.58 g, 20.0 mmol), and chloromethyl chloroformate (2.32 g, 18.0 mmol). Compound S1a was obtained as a colorless solid (4.22 g, 88%). ¹H NMR (400 MHz, CDCl₃) δ 5.69 (s, 2H), 4.30-4.26 (m, 2H), 3.48-3.46 (m, 2H), 2.87 (s, 3H), 1.41 (s, 9H). ¹³C{¹H} NMR (101 MHz, CDCl₃) δ 155.4, 153.4, 80.0, 72.4, 67.0, 47.8, 35.3, 28.5 (three carbons)

### Chloromethyl 3-((tert-butoxycarbonyl)(methyl)amino)propyl carbonate (S1b)

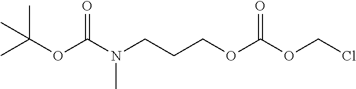 [SMILES: CN(CCCOC(=O)OCCl)C(=O)OC(C)(C)C]

According to GP A, chlorotnethyl carbonate S1b was prepared reacting 3-((tert-butoxycarbonyl)(methyl)amino)propanol (8.14 g, 43.0 mmol), pyridine (6.80 g, 86.0 mmol), and chloromethyl chloroformate (5.54 g, 43.0 mmol). Compound S1b was obtained as a colorless oil (10.3 g, 85%). ¹H NMR (400 MHz, CDCl₃) δ 5.67 (s, 2H), 4.18 (t, J=6.4 Hz, 2H), 3.26 (t, J=6.8 Hz, 2H), 2.79 (s, 3H), 1.86 (tt, J=6.7 Hz, J=6.4 Hz, 2H), 1.39 (s, 9H). ¹³C{¹H} NMR (101 MHz, CDCl₃) δ 155.5, 153.2, 79.5, 72.1, 66.7, 45,3, 34.3, 28.3 (three carbons), 27.0.

### General Procedure B (GP B) for the Formation of lodomethyl Carbonates (S2)

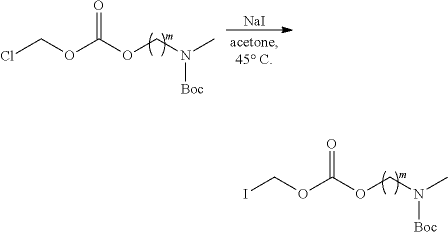

A round-bottom flask was charged with a solution of the respective chloromethyl carbonate in acetone (0.4 mL/mmol chloromethyl carbonate). NaI was added in portions. The reaction mixture was heated to 45° C. and it was allowed to stir for 12 h. Subsequently, the mixture was filtered and concentrated under reduced pressure. Information regarding the purification are given for each product.

### Iodomethyl 2-((tert-butoxycarbonyl)(methyl)amino)ethyl carbonate (S2a)

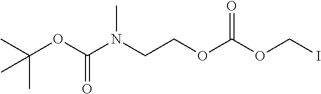 [SMILES: CN(CCOC(=O)OCI)C(=O)OC(C)(C)C]

According to GP B, iodomethyl carbonate S2a was prepared reacting chloromethyl carbonate S1a (2.68 g, 10.0 mmol) and Nal (4.50 g, 30.0 mmol). The crude product was solved in CH₃Cl (50 mL) and washed with coned Na₂CO_{3(aq)} (30.0 mL), coned NaHCO_{3(aq)} (3×20.0 mL), and water (30 mL), The organic layer was dried over Na₂SO₄ and concentrated under reduced pressure. Compound S2a was obtained as a colorless oil (3.38 g, 94%) without any further purifications. ¹H NMR (400 MHz, CDCl₃) δ 5.92 (s, 2H), 4.30-4.26 (m, 2H), 3.48-3.45 (m, 2H), 2.87 (s, 3H), 1.42 (s, 9H). ¹³C{¹H} NMR (101 MHz, CDCl₃) δ 155.7, 153.3, 80.1, 67.2, 47.8, 35,7, 34.1, 28.6 (three carbons)

### Iodomethyl 2-((tert-butoxycarbonyl)(methyl)amino)propyl carbonate (S2b)

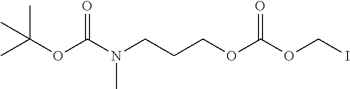 [SMILES: CN(CCCOC(=O)OCI)C(=O)OC(C)(C)C]

According to GP B, chloromethyl carbonate S2b was prepared reacting chloromethyl carbonate S1b (10.0 g, 35.5 mmol) and Nal (6.92 mg, 46.1 mmol). Purification by flash silica gel chromatography (DCM-n-hexane-Et₂O 5:4:1, R_{f}=0.58) afforded compound S2b as a yellowish oil (11.893 mg, 90%). ¹H NMR (400 MHz, CDCl₃) δ 5.83 (s, 2H), 4.11 (t, J=6.4 Hz, 2H), 3.19 (t, J=6.8 Hz, 2H), 2.73 (s. 3H), 1.79 (tt, J=6.7 Hz, J=6.4 Hz, 2H), 1.32 (s, 9H). ¹³C{¹H} NMR (101 MHz, CDCl₃) δ 155.4, 152.9, 79.3, 66.6, 45.3, 34.1. 28.3 (three carbons), 26.8.

### General Procedure C (GP C) for Alkylation/Acylation of 4(1H)-Quinolones (1)

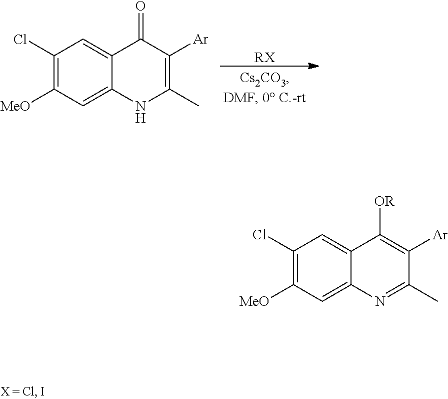

C-a) A round-bottom flask was charged with 4(1H)-quinolone (1.0 equiv) and Cs₂CO₃ (3.0 equiv) followed by addition of DMF (10.0-16.5 mL/mmol 4(1H)-quinolone). The suspension was cooled to 0° C. and stirred for 1 h. Subsequently, the respective alkylatinglacylating. agent (1.5-3.0 equiv) was added dropwise. The mixture was allowed to stir at room temperature for 18 h. The reaction was quenched with concd brine (5.0 mL/mmol 4(1H)-quinolone) and extracted with EA (5×6.0 mL/mmol 4(1H)-quinolone). The combined organic layers were washed with water (3×5.0 mL/mmol 4(1H)-quinolone) and brine (5.0 mL/mmol 4(1H)-quinolone), dried over Na₂SO₄ and concentrated under reduced pressure. The crude product was purified by flash silica gel chromatography.

C-b) A round-bottom flask was charged with a solution of the respective 4(1H)-quinolone (1.0 equiv) in DMF (5.0 mlimmol 4(1H)-quinolone). The stirred solution was cooled to 0° C. and Cs₂CO₃ (3.0 equiv) was added in portions. The cooling bath was removed, and the reaction was allowed to stir for 2 h at room temperature. The mixture was then recooled to 0° C. and the respective alkylating agent (1.5 equiv), dissolved in DMT (1.0 mL/mmol 4(1H)-quinolone), was added dropwise. The mixture was allowed to stir at room temperature for 10 h. The reaction was quenched with concd brine (5.0 miltrimol 4(1H)-quinolone) and extracted with EA (3×6.0 mL/mmol 4(1H)-quinolone). The combined organic layers were washed with water (5.0 mL/mmol 4(1H)-quinolone) and brine (5.0 mL/mmol 4(1H)-quinolone), dried over Na₂SO₄ and concentrated under reduced pressure. The crude product was purified by flash silica gel chromatography.

### 6-Chloro-7-methoxy-2-methyl-3-(o-tolyl)-4-((dimethylcarbamoyl)oxy)quinoline (2a)

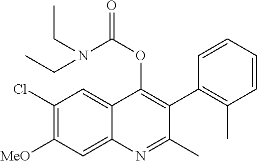 [SMILES: CCN(CC)C(=O)Oc1c(-c2ccccc2C)c(C)nc2cc(OC)c(Cl)cc12]

According to GP C-a, 4(1H)-quinolone 1c (200 mg, 0.64 mmol), Cs₂CO₃ (623 mg, 1.90 mmol), and diethyl carbamoylchloride (120 μL, 0.96 mmol, 1.5 equiv) were reacted in 6.4 mL DMF. Purification by flash silica gel chromatography (n-hexane-EA 2:1, R_{f}=0.30) afforded compound 2a as a colorless solid (171 mg, 65%). ¹H NMR (400 MHz, CDCl₃) δ 7.83 (s, 1H), 7.48 (s, 1H), 7.29-7.27 (m, 2H), 7.24-7.21 (m, 1H), 7.17 (d, J=7.3 Hz, 1H), 405 (s, 3H), 3.17-2.99 (m, 4H), 2.39 (s, 3H), 2.06 (s, 3H), 0.94 (t, J=7.1 Hz, 3H), 0.89 (t, J=7.1 Hz, 3H). ¹³C{¹H} NMR (126 MHz, CDCl₃) δ 160.3, 156,5, 152.7, 152.0, 149.0, 137.1, 134.5, 130.3, 130.2, 128.5, 126.4, 126.2, 124.5, 123.0, 118.0, 108.4, 56.8, 42.5, 42.1, 24.3, 20.0. 13,9, 13.3. HRMS (ESI-TOF) m/z: [M+H]⁺ calcd for C₂₃H₂₅ClN₂O₃ 413.1627; found 413.1616.

### 6-Chloro-7-methoxy-2-methyl-3-(o-tolyl)-4-((pyrrolidine-1-carbonyl)oxy)quinoline (2b)

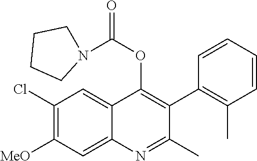 [SMILES: COc1cc2nc(C)c(-c3ccccc3C)c(OC(=O)N3CCCC3)c2cc1Cl]

According to GP C-a, 4(1H)-quinolone 1c (175 mg, 0.56 mmol), Cs₂CO₃(545 mg, 1.68 mmol), and 1-pyrrolidinecarbonyl chloride (185 μL, 1.68 mmol, 3.0 equiv) were reacted in 5.6 mL DMF. Purification by flash silica gel chromatography (n-hexane-EA 2:1, R_{f}=0.25) afforded compound 2b as a colorless solid (144 mg, 63%). ¹H NMR (500 MHz, CDCl₃) δ 7.93 (s, 1H), 7.47 (s, 1H), 7.29-7.28 (m, 2H), 7.25-7.21 (m, 3.0 Hz, 1H), 7.17 (d, J=7.3 Hz, 1H), 4.04 (s, 3H), 3.20-3.13, 2.92-2.87 (2m, 4H), 2.40 (s, 3H), 2.05 (s, 3H), 1.74-1.64 (m, 4H), ¹³C{¹H} NMR (126 MHz, CDCl₃) δ 160.2, 156.6, 152.2, 151.8, 149,0, 137.3, 134.5, 130.3, 130.2, 128.5, 126.2, 126.0, 124.5, 123.2, 118.0, 108.3, 56.8, 46.6, 46.4, 25.8, 25.1, 24.4, 20.0. HRMS (ESI-TOF) m/z: [M+H]⁺ calcd for C₂₃H₂₃ClN₂O₃ 411.147; found 411.146.

### 6-Chloro-7-methoxy-2-methyl-3-(o-tolyl)-4-(propionyloxy)quinoline (3)

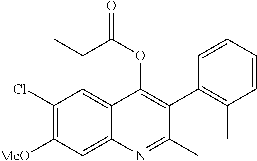 [SMILES: CCC(=O)Oc1c(-c2ccccc2C)c(C)nc2cc(OC)c(Cl)cc12]

According to GP C-a, 4(1H)-quinolone 1c (175 mg, 0.56 mmol), Cs₂CO₃ (545 mg, 1.68 mmol), and propionyl chloride (73.0 μL, 0.84 mmol, 1.5 equiv) were reacted in 8.0 mL DMF. Purification by flash silica gel chromatography (n-hexane-EA 2:1, R_{f}=0.29) afforded compound 3 as a yellowish solid (148 mg, 72%). ¹H NMR (400 MHz, CDCl₃) δ 7.74 (s, 1H), 7.49 (s, 1H), 7.31-7.29 (m, 2H), 7.25-7.22 (m, 1H), 7.09 (d, J=7.3 Hz, 1H), 4.05 (s, 3H), 2.41 (s. 3H), 2.34-2.18 (m, 2H), 2.04 (s, 3H), 0.88 (t, J=7.6 Hz, 3H). ¹³C{¹H} NMR (126 MHz, CDCl₃) δ 171.8, 160.3, 156.6, 151.3, 148.9, 137.0, 134.1, 130.4, 130.1, 128.6, 126.2, 126.0, 124.8, 122.5, 116.9, 108,5, 56.7, 27,6, 24.3, 19.8, 9.1. HRMS (ESI-TOF) m/z: [M+H]⁺ calcd for C₂₁H₂₀ClNO₃ 370, 1205; found 370, 1201.

### 6-Chloro-7-methoxy-2-methyl-3-(o-tolyl)-4-((ethoxycarbonyl)oxy)quinoline (4)

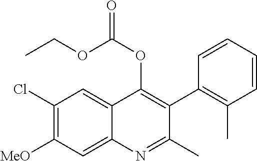 [SMILES: CCOC(=O)Oc1c(-c2ccccc2C)c(C)nc2cc(OC)c(Cl)cc12]

According to GP C-a, 4(1H)-quinolone 1c (175 mg, 0.56 mmol), Cs₂CO₃ (545 mg, 1.68 mmol), and ethyl chloroformate (106 μL, 1.12 mmol, 2.0 equiv) were reacted in 8.0 mL DMF. Purification by flash silica gel chromatography (n-hexane-EA 2:1, R_{f}=0.42) afforded compound 4 as a colorless solid (164 mg, 76%). ¹H NMR (400 MHz, CDCl₃) δ 7.87 (s, 1H), 7.48 (s, 1H), 7.31-7.28 (m, 2H), 7.25-7.22 (m, 1H), 7.13 (d, J=7.4 Hz, 1H), 4.06 (q, J=7.1 Hz, 2H), 4.03 (s, 3H), 2.40 (s, 3H), 2.05 (s, 3H), 1.10 (t, J=7.1 Hz, 3H). ¹³C{¹H} NMR (101 MHz, CDCl₃) δ 160.7, 156.8, 152.2, 150.8, 149.1, 137.2, 133.5, 130.6, 130.2, 128.8, 126.2, 125.9, 125.2, 122.4, 116.6, 108.6, 65.7, 56.8, 24.4, 19.9, 14.2. HRMS (ESI-TOF) m/z: [M+H]⁺ calcd for C₂₁H₂₀ClNO₄ 386.1154; found 386.1136.

### 6-Chloro-7-methoxy-2-methyl-3-(o-tolyl)-4-(((2-((tert-butoxycarbonyl)(methyl)amino)ethoxycarbonyl)oxy)methoxy)quinoline (5a)

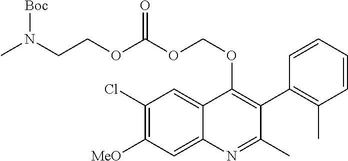 [SMILES: COc1cc2nc(C)c(-c3ccccc3C)c(OCOC(=O)OCCN(C)C(=O)OC(C)(C)C)c2cc1Cl]

According to GP C-a, 4(1H)-quinolone 1c (300 mg, 0.96 mmol), Cs₂CO₃ (935 mg, 2.87 mmol), and iodomethyl carbonate S2a (687 mg, 1.91 mmol, 2.0 equiv) were reacted in 16.0 mL DMF. Purification by flash silica gel chromatography (n-hexane-EA 1:1, R_{f}=0.48) afforded compound 5a as a colorless solid (354 mg, 68%). ¹H NMR (500 MHz, CDCl₃) δ 8.04 (s, 1H), 7.44 (s, 1H), 7.35-7.33 (m, 2H), 7.31-7.28 (m, 1H), 7.23 (d, J=7.4 Hz, 1H), 5.25 (d, J=6.1 Hz, 1H), 5.22 (d, J=6.1 Hz, 1H), 4.18-4.13 (m, 2H), 4.03 (s, 3H), 3.41-3.38 (m, 2H), 2.82 (s, 3H), 2.37 (s, 3H), 2.08 (s, 3H), 1.40 (s, 9H). ¹³ C{¹H} NMR (126 MHz, CDCl₃) δ 160.9, 156.7, 156.3, 156.0, 154.3, 149.0, 137.3, 134.6, 130.9, 130.7, 128.8, 126.5, 124.4, 124.1, 123.4, 118.0, 108.3, 91.1, 80.2, 66.7, 56.8, 47.9, 35.6, 28.7 (three carbons), 24.7, 20.1. HRMS (ESI-TOF) m/z: [M+H]⁺ calcd for C₂₈H₃₃ClN₂O₇ 545.2049; found 545.2074.

### 6-Chloro-7-methoxy-2-methyl-3-(o-tolyl)-4-(((3-((tert-butoxycarbonyl)(methyl)amino)propyloxycarbonyl)oxy)methoxy)quinoline (5b)

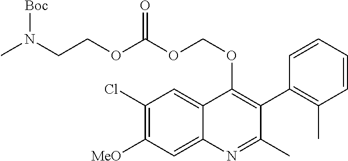 [SMILES: COc1cc2nc(C)c(-c3ccccc3C)c(OCOC(=O)OCCN(C)C(=O)OC(C)(C)C)c2cc1Cl]

According to GP C-a, 4(1H)-quinolone 1c (500 mg, 1.59 mmol), Cs₂CO₃ (1.56 g, 4.78 mmol), and iodomethyl carbonate S2b (1.19 g, 3.19 mmol, 2 equiv) in 0.06 molar DMF were reacted. Purification by flash silica gel chromatography (n-hexane-EA 2:1, R_{f}=0.22) afforded compound 5b as a colorless solid (678 mg, 76%). ¹H NMR (100 MHz, CDCl₃) δ 8.05 (s, 1H), 7.44 (s, 1H), 7.36-7.34 (m, 2H), 7.32-7.28 (m, 1H), 7.23 (d, J=7.3 Hz, 1H), 5.24 (d, J=6.1 Hz, 1H), 5.22 (d, J=6.1 Hz, 1H), 4.08 (t, J=6.5 Hz, 2H), 4.04 (s, 3H), 3.21 (t, J=6.1 Hz, 2H), 2.79 (s, 3H), 2.38 (s, 3H), 2.09 (s, 3H), 1.84-1.78 (m, 2H), 1.41 (s, 9H). ¹³C{¹H} NMR (126 MHz, CDCl₃) δ 160.9, 156.7, 156.3, 156.0, 154.5, 149.0, 137.3, 134.6, 131.0, 130.7, 128.9, 126.6, 124.4, 124.1, 123.4, 118.1, 108.1, 90.8, 79.6, 66.5, 56.6, 45.6, 34.4, 28.5 (three carbons), 27.2, 24.5, 19.9. HRMS (ESI-TOF) m/z: [M+H]⁺ calcd for C₂₉H₃₅ClN₂O₇ 559.2206; found 559.2197.

### 6-Chloro-7-methoxy-2-methyl-3-(4-(trifluoromethyl)phenyl)-4-(((2-((tert-butoxycarbonyl)(methyl)amino)ethoxycarbonyl)oxy)methoxy)quinoline (5c)

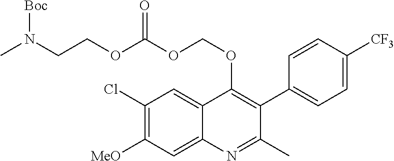 [SMILES: COc1cc2nc(C)c(-c3ccc(C(F)(F)F)cc3)c(OCOC(=O)OCCN(C)C(=O)OC(C)(C)C)c2cc1Cl]

According to GP C-a, 4(1H)-quinolone 1d (300 mg, 0.82 mmol) Cs₂CO₃(797 mg, 2.45 mmol), and iodomethyl carbonate S2a (586 mg, 1.63 mmol, 2 equiv) were reacted in 13.7 mL DMF. Purification by flash silica gel chromatography (n-hexane-EA 1:1, R_{f}=0.32) afforded compound 5c as a colorless solid (340 mg, 70%). ¹H NMR (500 MHz, CDCl₃) δ 8.01 (s, 1H), 7.75 (d, J=8.1 Hz, 2H), 7.52 (d, J=8.0 Hz, 2H), 7.43 (s, 1H), 5.27 (s, 2H), 4.12-4.07 (m, 2H), 4.03 (s, 3H), 3.40-3.36 (m, 2H), 2.80 (br s, 3H), 2.46 (s, 3H), 1.40 (s, 9H). ¹³C{¹H} NMR (126 MHz, CDCl₃) δ 159.7, 157.0, 156.6, 155.5, 154.2, 149.3, 139.1, 131.1 (two carbons), 130.5 (q, J=32.7 Hz), 126.0 (q, J=3.6 Hz; two carbons), 124.9, 124.3 (q, J=272.0 Hz), 124.2, 123.2, 117.6, 108.4, 91.5, 80.2, 66.8, 56.8, 47.9, 35.5, 28.6 (three carbons), 25.2. ¹⁹F NMR (376 MHz, CDCl₃) δ−62.6 (three fluorines). HRMS (ESI-TOF) m/z: [M+H]⁺ calcd for C₂₈H₃₀ClF₃N₂O₇ 599.1766; found 599.1784.

### 6-Chloro-7-methoxy-2-methyl -3-(4-(tiifluoromethyl)phenyl)-4-(((3-((tert-butoxycarbonyl)(methyl)amino)propyloxycarbonypoxy)methoxy)quinoline (5d)

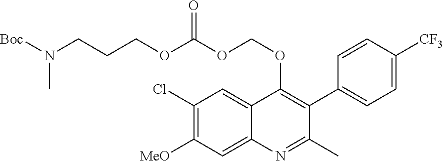 [SMILES: COc1cc2nc(C)c(-c3ccc(C(F)(F)F)cc3)c(OCOC(=O)OCCCN(C)C(=O)OC(C)(C)C)c2cc1Cl]

According to GP C-a, 4(1H)-quinolone 1d (300 mg, 0.82 mmol), Cs₂CO₃(797 mg, 2.45 mmol), and iodomethyl carbonate S2b (609 mg, 1.63 mmol, 2 equiv) were reacted in 13.7 mL, DMF. Purification by flash silica gel chromatography (n-hexane-EA 1:1, R_{f}=0.39) afforded compound 5d as a colorless solid (339 mg, 68%). ¹H NMR (500 MHz, CDCl₃) δ 8.01 (s, 1H), 7.75 (d, J=8.1 Hz, 2H), 7.52 (d, J=8.0 Hz, 2H), 7.42 (s, 1H), 5,26 (s, 2H), 4.03 (s, 3H), 4.02 (t, J=6.5 Hz, 2H), 3.20-3.17 (m, 2H), 2.78 (s, 3H), 2.46 (s, 3H), 1.79-1.76 (m, 2H), 1.40 (s, 9H). ¹³ C{¹H} NMR (126 MHz, CDCl₃) δ 159.7, 157.0, 156.7, 155.9, 154.3, 149.3, 139.2, 131.1 (two carbons), 130.5 (q, J=32.8 Hz), 126.0 (q, J=3.6 Hz; two carbons), 124.9, 124.3 (q, J=272.2 Hz), 124.1, 123.3, 117.7, 108.4, 91.3, 79.8, 66.2, 56.8, 45.46, 34.7, 28.7 (three carbons), 27.3, 25.2. ¹⁹F NMR (376 MHz, CDCl₃) δ−62.6 (three fluorines). HRMS (ESI-TOF) [M+H]⁺ calcd for C₂₉H₃₂ClF₃N₂O₇ 613.1923; found 613.1931.

### 6-Chloro-7-methoxy-2-methyl-3-(2-fluoro-4-(4-(trifluoromethoxy)phenoxy)phenyl)-4-(((3-((tert-butoxycarbonyl)(methyl)amino)propyloxycarbonyl)oxy)methoxy)quinoline (5e)

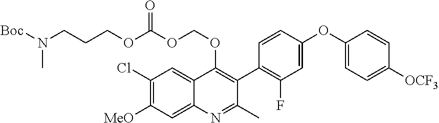 [SMILES: COc1cc2nc(C)c(-c3ccc(Oc4ccc(OC(F)(F)F)cc4)cc3F)c(OCOC(=O)OCCCN(C)C(=O)OC(C)(C)C)c2cc1Cl]

According to GP C-b, 4(1H)-quinolone 1b (1.50 g, 3.04 mmol), Cs₂CO₃ (2.97 g, 9:12 mmol), and iodomethyl carbonate S2b (1.70 g, 4.56 mmol) were reacted. Purification by twofold flash silica gel chromatography (1. Et₂O-toluene-DCM 4:4:2, R_{f}=0.42; 2, Et₂O-DCM-toluene-n-hexane 5:2:2:1, R_{f}=0.32) afforded compound 5e as a colorless solid (1.79 g, 80%). ¹H NMR (400 MHz, CDCl₃) δ 8.01 (s, 1H, m-CH_{Ar}(N)), 7.46 (s, 1H, o-CH_{Ar}(N)), 7.32-7.28 (m, 3H, m-CH_{Ar}(F), 2× m-CH_{Ar}(OCF₃)), 7.18-7.16 (m, 2H, 2× o-CH_{Ar}(OCF₃)), 6.93 (dd, ³J_{HH}=8.5 Hz, ⁴J_{HH}=1.8 Hz. 1H, p-CH_{Ar}(F)), 6.88 (dd, ³J_{HF}=10.5 Hz, ⁴J_{HH}=1.8 Hz, 1H, o-CH_{Ar}(F)), 5.44 (d, ²J_{HH}=6.0 Hz, 1H, OCH₂O), 5.31 (d, ²J_{HH}=6.0 Hz, 1H, OCH₂O), 4.12 (t, ³J_{HH}=6.5 Hz, 2H, OCH₂CH₂CH₂N), 4.06 (s, 3H, OCH₃), 3.26-3.23 (m, 2H, OCH₂CH₂CH₂N), 2.82 (s, 3H, NCH₃), 2.52 (s, 3H, C_{q.Ar}CH₃), 1.86-1.83 (m, 2H, OCH₂CH₂CH₂N), 1.43 (s, 9H, C(CH₃)₃). ¹³ C{¹H} NMR (101 MHz, CDCl₃) δ 160.4 (d, ¹J_{CF}=248.3 Hz, C_{q.Ar}F), 160.3 (C_{Ar}CH₃), 158.8 (d, ³J_{CF}=10.2 Hz, m-C_{q.Ar}(F)), 157.5 (C_{q.Ar}OCH₂), 156.6 (C_{q,Ar}OCH₃), 155.5 (NC(O)O), 154.2 (p-C_{q,Ar}(OCF₃)), 153.9 (OC(O)O), 149.0 (p-C_{q.Ar}(Cl)), 145.3 (q, ³J_{CF}=1.6 Hz, C_{q.Ar}OCF₃), 132.9 (d, ³J_{CF}=5.1 Hz, m-CH_{Ar}(F)), 124.2 (C_{q.Ar}Cl), 122.9 (m-CH_{Ar}(OCH₃)), 122.8 (2×m-CH_{Ar}(OCF₃); two carbons), 120.8 (2×o-CH_{Ar}(OCF₃); two carbons), 120.4 (q, ¹J_{CF}=259.2 Hz, CF₃), 118.4 (o-C_{q.Ar}(CH₃)), 117.3 (m-C_{q.Ar}(Cl)), 117.1 (d, ²J_{CF}=17.2 Hz, o-C_{q.Ar}(F)), 114.1 (d, ⁴J_{CF}=3.0 Hz, p-CH_{Ar}(F)), 108.0 (o-CH_{Ar}(OCH₃), 106.2 (d, ²J_{CF}26 Hz, o-CH_{Ar}(F)), 91.1 (OCH₂O), 79.3 (C(CH₃)₃)), 66.3 (OCH₂CH₂CH₂N), 56.3 (OCH₃), 45.3 (OCH₂CH₂CH₂N), 34.2 (NCH₃), 28,2 (C(CH₃)₃), 26.8 (OCH₂CH₂CH₂N), 24.1 (C_{q.Ar}CH₃). ¹⁹F NMR (376 MHz, CDCl₃) δ−59.2 (OCF₃; three fluorines), −112.1 (dd, ³J_{HF}=10.4 Hz, ⁴J_{HF}=8.2 Hz, C_{q.Ar}F).

### General Procedure (GP D) for Deprotection of Doc-Protected Prodrugs (5)

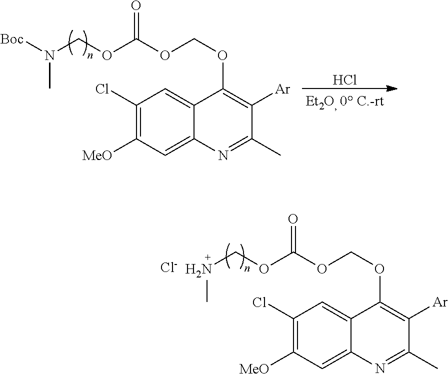

D-a) A round-bottom flask was charged with a solution of the respective Boc-protected 4-alkoxyquinolin in Et₂O (10.0 mL/mmol 4-alkoxyquinolin). The solution was cooled to 0° C. and in situ generated HCl_{(g)} was bubbled through for 10 min. After stirring for 10 min at 0° C. the cooling bath was removed, and the reaction was allowed to stir for additional 10 min at room temperature upon precipitation of the HCl salt. The mixture was then concentrated under reduced pressure. The pure product was obtained without any further purification.

D-b) A round-bottom flask was charged with the respective Boc-protected 4-alkoxyquinolin. At 0° C. HCl (2 M in Et₂O; 25.0 mL/mmol 4-alkoxyquinoli ewas added. After stirring for 30 min at 0° C. the cooling bath was removed, and the reaction was allowed to stir for additional 10 h at room temperature upon precipitation of the HCl salt. The mixture was then concentrated under reduced pressure. The pure product was obtained without any further purification.

### 6-Chloro-7-methoxy-2-methyl-3-(o-tolyl)-4-(((2-(methyl ammonio)ethoxycarbonyl)oxy)methoxy)quinoline, chloride salt (6a)

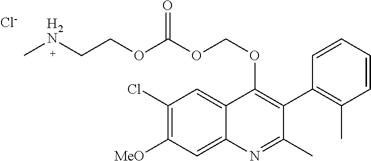

According to GP D-a, Boc-p ected 4-alkoxyquinoline 5a (25 mg, 0.05 mmol) was deprotected and the HCl salt 6a was obtained as a colorless solid (21 mg, 95%). NMR (500 MHz, DMSO-d₆) δ 9.20 (br s, 2H), 8.28 (s, 1H), 8.03 (s, 1H), 7.47-7.43 (m, 2H), 7.40-7.37 (m, 1H), 7.35-7.34 (m, 1H), 5.44 (d, J=6.1 Hz, 1H), 5.40 (d, J=6.1 Hz, 1H), 4.28 (t, J=5.2 Hz, 2H), 4.08 (s, 3H), 3.16-3.12 (m, 2H), 2.51-2.49 (m, 6H), 2,06 (s, 3H). ¹³C{¹H} NMR (126 MHz, DMSO-d₆) δ 160.1, 159.0, 157.9, 152.9, 141.9, 136.9, 131.2, 130.6, 130.5, 129.4, 126.4, 125.4, 124.2, 123.7, 116.9, 102.3, 91.1, 63.6, 57.2, 46.4, 32.5, 20.7, 19.2. HRMS (ESI-TOF) m/z: [M]⁺ calcd for C₂₃H₂₆ClN₂O₅⁻445.1525; found 445.151.

### 6-Chloro-7-methoxy-2-methyl-3-(o-tolyl)-4-(((3-(methyl ammonio)propyloxycarbonyl)oxy)methoxy)quinoline, chloride salt (6b)

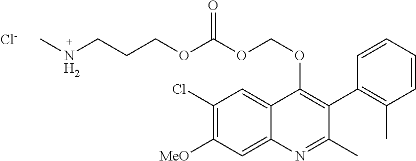 [SMILES: [Cl-] ; CNCCCOC(=O)OCOc1c(-c2ccccc2C)c(C)nc2cc(OC)c(Cl)cc12]

According to GP D-a, Boc-protected 4-alkoxyquinolin 5b (200 mg, 0.36 mmol) was deprotected and the HCl salt 6b was obtained as a colorless solid (164 mg, 93%). ¹H NMR (500 MHz, DMSO-d₆) δ 8.85 (br s, 2H), 8.19 (s, 1H), 7.86 (s, 1H), 7.46-7.42 (m, 2H), 7.39-7.36 (m, 1H), 7,31 (d, J=7.4 Hz, 1H), 5.40 (d, J=6.2 Hz, 1H), 5.38 (d, J=6.2 Hz, 1H), 4.08 (s, 3H), 4.06 (t, J=6.5 Hz, 2H), 2.89-2.83 (m, 2H), 2.51 (t, J=3.4 Hz, 3H), 2.44 (s, 3H), 2.05 (s, 3H), 1.93-1.88 (m, 2H), ¹³{¹H} NMR. (101 MHz, DMSO-d₆) δ 159.4, 158.5, 153.2, 143.6, 136.6, 130.5, 130.3, 129.0, 126.3, 124.2, 123.2, 116.9, 90.9, 65.4, 57.1, 44.9, 32.3, 24.6, 19.3. HRMS (ESI-TOF) m/z: [M]⁺ calcd for C₂₄H₂₈ClN₂O₅⁺459,1681; found 459.1689. (Signals corresponding to the carbons C_{q.Ar}CH₃, o-CH_{Ar}(OCH₃), C_{q-Ar}OCH₂, o-C_{q.Ar}(CH₃) were too broad to be observable.)

### 6-Chloro-7-methoxy-2-methyl-3-(4-(triftuoromethyl)phenyl)-4-(((2-(methyl ammonio)ethoxycarbonyl)oxy)methoxy)quinoline, chloride salt (6c)

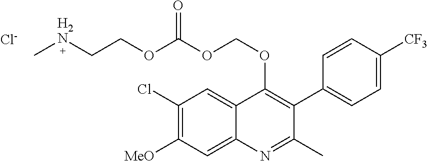

According to GP D-a, Boc-protected 4-alkoxyquinolin 5c (60 mg, 0.10 mmol) was deprotected and the HCl salt 6c was obtained as a colorless solid (50 mg, 93%). ¹H NMR (500 MHz, DMSO-d₆) δ 9.22-9.19 (m, 2H), 8.30 (s, 1H), 8.00 (s, 1H), 7.96 (d, J=8.2 Hz, 2H), 7.71 (d, J=8.0 Hz, 2H), 5.53 (s, 2H), 4.27-4.25 (m, 2H), 4.09 (s, 3H), 3.13-3.13 (m, 2H), 2.59 (s, 3H), 2.52-2.50 (m, 3H). ¹³C{¹H} (126 MHz, DMSO-d₆) δ 158,7, 157.8, 152.8, 136.6, 131.1 (two carbons), 129.1 (q, J=31.6 Hz), 125.7 (q, J=3.5 Hz; two carbons), 124.7, 124.2 (q, J=272.2 Hz), 123,6, 116.8, 91.9, 63.6, 57.2, 46.6, 32.5, 21.5. ¹⁹F NMR (376 MHz, DMSO-d₆) δ−61.0 (three fluorines). HRMS (ESI-TOF) m/z: [M]⁺ calcd for C₂₃H₂₃ClF₃N₂O₅⁺ 499.1242; found 499.126, (Signals corresponding to the carbons C_{q.Ar}CH₃, o-CH_{Ar}(OCH₃),p-C_{q.Ar}(Cl), o-C_{q.Ar}(CH₃) were too broad to be observable.)

### 6-Chloro-7-methoxy-2-methyl-3-(4-(trifluoromethyl)phenyl)-4-(((3-(methyl ammonio)propyloxycarbonyl)oxy)methoxy)quinoline, chloride salt (6d)

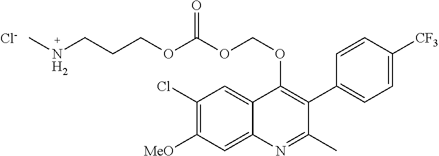 [SMILES: [Cl-] ; CNCCCOC(=O)OCOc1c(-c2ccc(C(F)(F)F)cc2)c(C)nc2cc(OC)c(Cl)cc12]

According to GP D-a, Boc-protected 4-alkoxyquinolin 5d (65 mg, 0.11 mmol) was deprotected and the HCl salt 6d was obtained as a colorless solid (56 mg, 95%). ¹H NMR (500 MHz, DMSO-d₆) δ 9.07-9.03 (m, 2H), 8.26 (s, 1H), 7.98 (s, 1H), 7.94 (d, J=8.2 Hz, 2H), 7.71 (d, J=8.0 Hz, 2H), 5.50 (s, 2H), 4.09 (s, 3H), 4.04 (t, J=6.3 Hz, 2H), 2.87-2.81 (m, 2H), 2.58 (s, 3H), 2.49 (t, J=5.5 Hz, 3H), 1.96-1.87 (m, 2H). ¹³ C{¹H} NMR (126 MHz, DMSO-d₆) δ 158.7, 157.7, 153.1, 136.6, 131.1 (two carbons), 129.0 (q, J=31.6 Hz), 125.6 (q, J=3.3 Hz; two carbons), 124.8, 124.1 (q, J=272.2 Hz), 123.6, 116.9, 91.7, 65.4, 57.2, 45.8, 32.2, 24.5, 21.7. ¹⁹F NMR (376 MHz, DMSO-d6) δ−61.1 (three fluorines). HRMS (ESI-TOF) m/z: [M]⁺ calcd for C₂₄H₂₅ClF₃N₂O₅⁺ 513.1399; found 513.1397. (Signals corresponding to the carbons C_{q.Ar}CH₃, o-CH_{Ar}(OCH₃), p-C_{q.Ar}(Cl), o-C_{q.Ar}(CH₃) were too broad to be observable.)

### 6-Chloro-7-methoxy-2-methyl-3-(2-fluoro-4-(4-(trifluoromethoxy)phenoxy)phenyl)-4-(((3-(methyl ammonio)propyloxycarbonyl)oxy)methoxy)quinoline, chloride salt (6e)

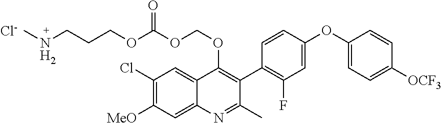 [SMILES: [Cl-] ; CNCCCOC(=O)OCOc1c(-c2ccc(Oc3ccc(OC(F)(F)F)cc3)cc2F)c(C)nc2cc(OC)c(Cl)cc12]

According to GP D-a, Boc-protected 4-alkoxyquinolin 5e (300 mg, 0.41 mmol) was deprotected and the HCl salt fie was obtained as a colorless solid (271 mg, 98%). ¹H NMR (500 MHz, DMSO-d₆) δ 9.30-9.24 (m, 2H, NH₂⁻), 8.31 (s, 1H, m-CH_{Ar}(N)), 8.07 (s, 1H, o-CH_{Ar}(N)), 7.57 (dd, ³J_{HH}=8.5 Hz, ⁴J_{HF}=8.4 Hz, 1H, m-CH_{Ar}(F)), 7.50-7.46 (m, 2H, 2×m-CH_{Ar}(OCF₃)), 7.33-7.30 (m, 2H, 2×o-CH_{Ar}(OCF₃)), 7.21 (dd, ³J_{HF}=10.7 Hz, ⁴J_{HH}=2.4 Hz, 1H, o-CH_{Ar}(F)), 7.07 (dd, ³J_{HH}=8.5 Hz, ⁴J_{HH}=2.3 Hz, 1H, p-CH_{Ar}(F)), 5.61 (d, ²J_{HH}=6.5 Hz, 1H, OCH₂O), 5.58 (d, ²J_{HH}=6.5 Hz, 1H, OCH₂O), 4.10 (t, ³J_{HH}=6.6 Hz, 2H, OCH₂CH₂CH₂N), 4.09 (s, 3H, OCH₃), 2.85 (tt, ³J_{HH}=7.3 Hz, ³J_{HH}=6.1 Hz, 2H, OCH₂CH₂CH₂N), 2.47 (t, ³J_{HH}=5.5 Hz, 3H, NCH₃), 2.68 (s, 3H, C_{q.Ar}CH₃), 1.96 (tt, ³J_{HH}=7.3 Hz, ³J_{HH}=6.6 Hz, 2H, OCH₂CH₂CH₂N) ¹³C{¹H} NMR (101 MHz, DMSO-d₆) δ 162.1 (C_{q.Ar}OCH₂), 159.9 (d, ¹J_{CF}=247.0 Hz, C_{q.Ar}F), 158.9 (C_{Ar}CH₃), 158.8 (d, ³J_{CF}=11.5 Hz, m-C_{q.Ar}(F)), 158.4 (C_{q.Ar}OCH₃), 154.3 (p-C_{q.Ar}(OCF₃)), 153.1 (OC(O)O), 144.5 (q, ³J_{CF}=1.4 Hz, C_{q.Ar}OCF₃), 141.6 (p-C_{q.Ar}(Cl)), 133.6 (d, ³J_{CF}=4.2 Hz, m-CH_{Ar}(F)), 125.9 (C_{q.Ar}Cl), 123.8 (m-CH_{Ar}(OCH₃)), 123.2 (two carbons, 2×m-CH_{Ar}(OCF₃)), 120.9 (two carbons, 2×o-CH_{Ar}(OCF₃)), 120.1 (q, 256.0 Hz, CF₃), 119.5 (O-C_{q.Ar}(CH₃)), 116.9 (m-C_{q.Ar}(Cl)), 114.8 (d, ⁴J_{CF}=2.4 Hz, p-CH_{Ar}(F)), 113.9 (d, ²J_{CF}=17.7 Hz, o-C_{q.Ar}(F)), 102.0 (o-CH_{Ar}(OCH₃), 106.6 (d, ²J_{CF}=25.9 Hz, o-CH_{Ar}(F)), 91.7 (OCH₂O), 65.7 (OCH₂CH₂CH₂N), 57.3 (OCH₃), 44.9 (OCH₂CH₂CH₂N), 32.2 (NCH:₃), 24.6 (OCH₂CH₂CH₂N), 20.2 (C_{q.Ar}CH₃). ¹⁹F NMR (376 MHz, CDCl₃) δ−59.2 (OCF₃; three fluorines), −112.1 (dd, ³J_{HF}=10.4 Hz, ⁴J_{HF}=8.2 Hz, C_{q.Ar}F).

In Vivo Pharmacokinetics in Mice

Dosing

Compounds 1b and its prodrug 6e were administered as a single dose (10 mg/kg) in 0.5% HEC using freshly prepared solution. Parent compound 1d and its prodrugs 6c and 6d were administered as a single dose of 50 mg/kg using the same vehicle. The blood was collected in prepared 5 ml syringe containing heparin via cardiac puncture and put into 15 mL conical tube on ice. Five mice were used per one time point at 0.5 h, 1 h, 2 h, 4 h, 8 h and 24 h post-rearmament plus 2 mice as a control (not-treated). The blood was then centrifuged for 5 min at 4000 rpm and plasma supernatant collected while avoiding the whole blood pellet at the bottom of the tube. The plasma was stored at −80° C. until the day of LC/MS-MS analysis.

LC/ISIS-MS Analysis

Compound concentrations were quantitated in plasma samples by LC/MS-MS using an Agilent triple quadrupole instrument. Chromatographic separation was conducted using an Agilent HPLC with Phenomenex Kinetex C18 column (2.6 μm particle size, 50-4.6 mm i.d.) equipped with a Phenomenex Security Guard column. The mobile phase (1.25 mL/min) consisted of 0.05% formic acid in water and 0.05% formic acid in acetonitrile mixed using a linear gradient over 7 minutes. The injection volume was 10 μL and elution of analytes was confirmed by multiple-reaction monitoring (MRM) using 6-chloro-7-methoxy-2-methyl-3-phenylquinolin-4(1H)-one²⁰ as the internal standard. Plasma samples and calibration standards were prepared by protein precipitation with acetonitrile (3 parts acetonitrile to 1 part plasma), followed by centrifugation and analysis of the supernatant. Sample concentrations were determined by comparison to calibration standards prepared in blank plasma and assayed using the same conditions. The analytical lower limit of quantitation in plasma was typically 0.75-1.00 nM and accuracy, precision and recovery were within acceptable limits.

Calibration Curves

The following concentrations points were prepared using commercially available Balb/c mouse plasma (prepared from whole blood collections from normal healthy mice) and stored at −80° C. 10 μM stock of test compound in mouse plasma (prepared from a 10 mM stock solution in DMSO): 1000 nM (100 μL, stock (10 μM)+900 μL plasma), 500 nM (50 μL stock (10 μM)+950 μL plasma), 100 nM (10 μL stock (10 μM)+990 μL plasma), 50 nM (50 μL stock (1 μl)+950 μL plasma), 25 nM (25 μL stock (1 μM)+975 μL plasma), 10 nM (10 μL stock (1 μM)+990 μL plasma), 5 nM (5 μL stock (1 μM)+995 μL plasma), and 1 nM (1 μL stock (1 μM)+999 μL plasma).

Sample Preparation

The plasma was first warmed up on ice for about 1 hour. Then 50 μL of plasma sample was precipitated with 150 μL of cold acetonitrile with internal standard (133 nM) P4Q-95²⁰) following by centrifugation at 4,000 rpm for 5 min at 4° C. and transfer˜150 μL of the supernatant to LCMS vial. The samples were analyzed using Agilent triple quadruple instrument in triplicates (5 mice×3 injections for each time point). The chemical structure of P4Q-95²⁰ is shown below:

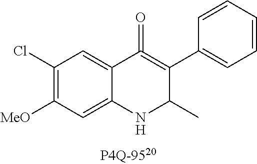

The calibration curve for 1d was linear in 1 to 500 nM range so samples were diluted if needed to fit this linearity and then concentration recalculated accordingly. Calibration curve was run each time following the actual PK measurements and used for that particular compound.

Dose Linear of Amino AOCOM Ether Prodrug

Previous in vivo efficacy and pharmacokinetic studies with frontrunner 4(1H)-quinolones 1a and 1b failed to generate high fidelity dose linearity relationships and were thus unsuitable to adequately assess safety margins. For example, even though acceptable oral bioavailability for 1a and 1b was observed at therapeutically relevant doses, an inverse correlation between oral bioavailability and dose supports the notion that absorption was limited by poor aqueous solubility.⁵⁰

Prodrug 6d was orally tested for in vivo efficacy at four doses (0.3 mg/kg, 1.0 mg/kg, 3.0 mg/kg, and 10 mg/kg) in 0.5% aqueous HEC solution. Reduction in parasitemia on day 6 PI increased from 11% to 51% in a nearly linear dose dependent manner (FIG. 6) highlighting the utility of the solubili zing prodrug moiety.

Amino AOCOM Prodrug of ICI56,780

To demonstrate the versatility of the developed prodrug approach, the amino AOCOM prodrug moiety was installed in ICI56,780(7), another poorly soluble 4(1H)-quinolone with antimalarial activity. Prodrug 8 was prepared starting from 4(1H)-quinolone ester 7 using conditions outlined in FIG. 4 and tested for in vivo efficacy. The chemical structures of ICI 56,780 (7) and its AOCOM prodrug 8 are shown below:

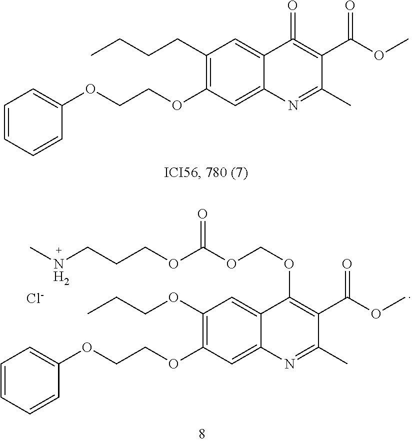

Significant improvements in antimalarial activity were observed for prodrug 8 over reference compound 7 (Table 4). At 3 doses of 10 mg/kg of 7, no activity was observed on day 6 PI, whereas prodrug 8 at the same doses reduced parasitemia by 79%.

TABLE 4
In vivo efficacy and PK parameters of 7 and its amino AOCOM ether
prodrug 8.

| | 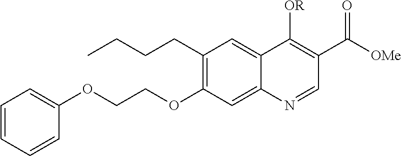 | |
|---|---|---|
| No | 7 | 8 |
| R | —H | 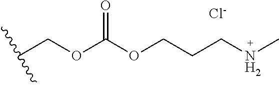 |
| MW | 395.46 | 577.07 |
| dose | 10^{a} | 10^{a} |
| [mg/kg] | | |
| suppression | <1 | 79 |
| [%] day 6 | | |
| PE^{b} | | |
| day of | 13 | 16 |
| death^{c}(avg) | | |
| Cₘₐₓ | n.d. | 0.5 |
| [μmol/L] | | |
| tₘₐₓ[min] | n.d. | 60 |
| AUC_{0→24} | n.d. | 1.438 |
| [min · μmol/ | | |
| L] | | |
| V_{d}[L/kg] | n.d. | 34 |
| CL | n.d. | 12050 |
| [mL · kg/min] | | |
| t_{1/2 apparent}[h] | n.d. | 0.03 |

^{a}Oral administration as three daily doses (formulated in 0.5% aqueous HEC) on days 3-5 PI for in vivo efficacy and for PK analysis.
^{b}Percent suppression as compared to untreated control animals.
^{c}Untreated mice survived 13 days.

Since prodrug 8 displayed suppressive antimalarial activity in vivo and parent ICI56,780 (7) was inactive in the same assay, we performed PK experiments for the prodrug only. Prodrug 8 reached a Cₘₐₓ of 0.5 μM at 1 h, although with significantly lower AUC relative to 6e. Importantly, the pharmacokinetics and in vivo efficacy data underscore the utility of the amino AOCOM prodrug, as prodrug 8 at a three 10 mg/kg oral doses showed 79% suppression of parasitemia on day 6 PI, which is in stark contrast to parent compound 7 completely lacking in in vivo efficacy.

In summary, the prodrug moiety design comprises an amino group, which in a pH-dependent manner not only improves aqueous solubility but also initiates the prodrug's release mechanism rendering the prodrug activation to be completely independent of any enzymatic activity. The synthesis of the amino AOCOM prodrug moiety is straightforward as it can generally be attached to any parent compound containing an appropriate heteroatom. For example, the amino AOCOM prodrug moiety was installed in analogues 6a-6e of antimalarial 3-aryl-4(1H)-quinolone series, whose clinical development was halted due to poor oral bioavailability. Significant improvements of exposure and in vivo efficacy was observed for all amino AOCOM 4(1H)-quinolones prodrugs with 6e producing single dose cures at a low oral dose of 3 mg/kg. This in combination with the previously reported potent in vivo efficacy against the liver stages (with single oral dose of ≤10.1 mg/kg) and the stages that are crucial to disease transmission (with single oral dose of 0.1 mg/kg), restore the 3-aryl-4(1H)-quinolones as an attractive class of antimalarials with potential for clinical development.

Embodiments of the present disclosure further relate to any one or more of the following paragraphs:

1. A compound or a salt thereof, the compound comprising a Formula (I):

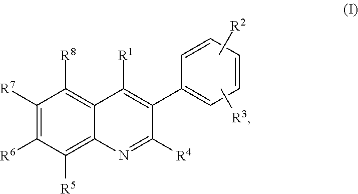

wherein R¹ is selected from H, F, Cl, Br, I, CN, CH₃, CF₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalken:v1, c:vcloalkynyl, h:vdroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbatnoyl, trifluoromethyl, phenoxy, benzyloxy, phosphoric acid, phosphate ester, sulfonic acid (—SO₃H), sulfonate ester, sulfonamide, carbamate, alkyltriphenylphosphonium,

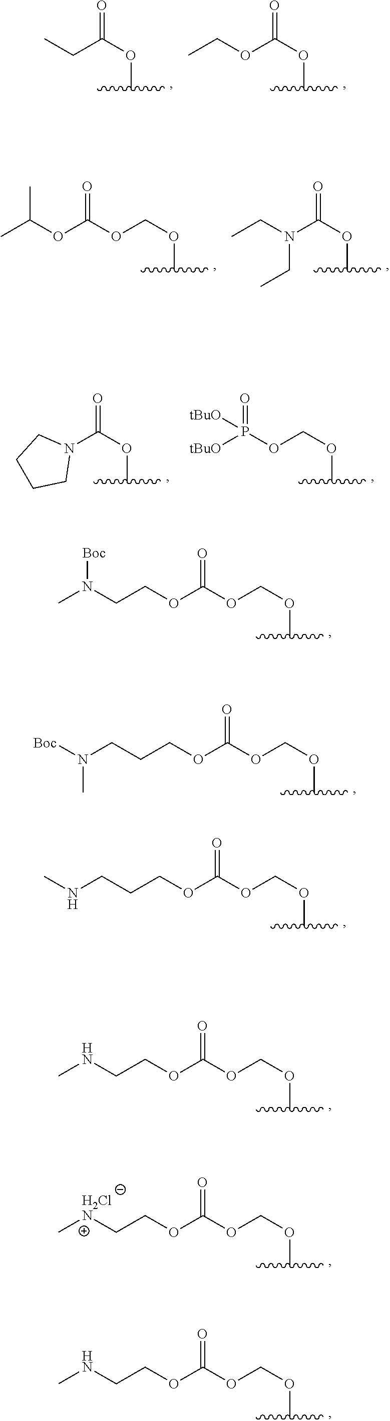

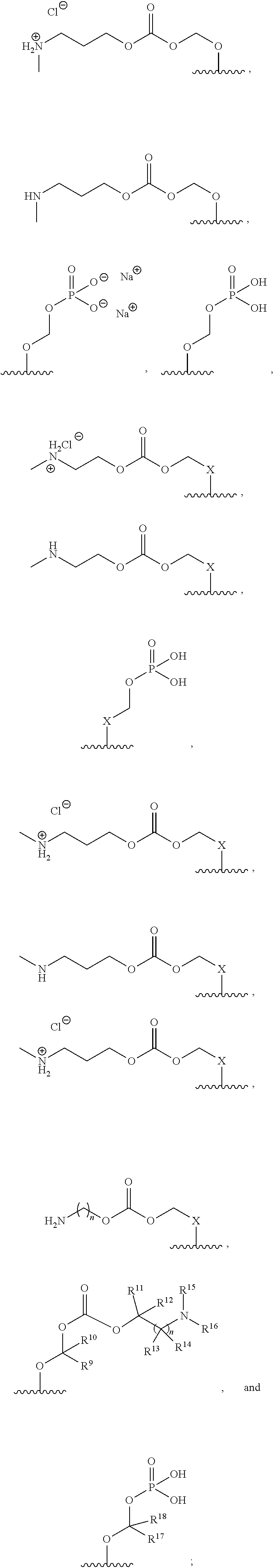

wherein R², R², R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently selected from H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium, and

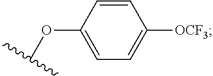

wherein X is selected from NH, NR¹⁹, oxygen, sulfur, and selenium, wherein is selected from the group R¹⁹ H, F, Cl, Br, I, CN, CH₃, CF₃, OCH₃, alkyl, halogenated alkyl, heteroalkyl, alkenyl, alkynyl, aryl, arylalkyl,aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbatnoyl, trifluoromethyl, phenoxy, benzyloxy, phosphonic acid, phosphate ester, sulfonic acid (—SO₃H), sulfonate ester, sulfonamide, and carbamate, alkyluiphenylphosphonium; and

wherein n is 1, 2, 3, or 4.

2. The compound according to paragraph 1, wherein the compound comprises a formula:

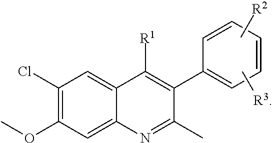

3. The compound according to paragraph 1 or 2, wherein the compound comprises a formula:

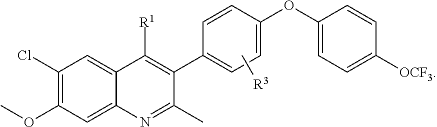

4. The compound any one of paragraphs 1 to 3, wherein the compound comprises a formula:

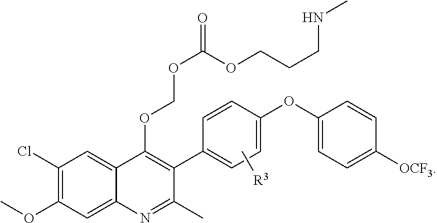

5. The compound any one of paragraphs 1 to 4, wherein the compound comprises a formula:

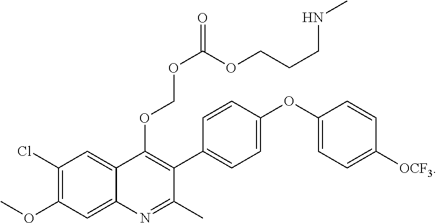 [SMILES: CNCCCOC(=O)OCOc1c(-c2ccc(Oc3ccc(OC(F)(F)F)cc3)cc2)c(C)nc2cc(OC)c(Cl)cc12]

6. A composition, the composition comprising one or more compounds of any one of paragraphs 1 to 5 and pharmaceutically acceptable carrier.

7. A method of treating malaria, the method comprising administering a composition comprising one or more compounds of any one of paragraphs 1 to 6.

8. The method of treating malaria according to any one of paragraphs 1 to 7, wherein the compounds are present in the composition from about 1 mg/kg to about 30 mg/kg.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility. It is therefore intended by the appended claims to cover any and all such applications, modifications and embodiments within the scope of the present invention.

As used herein, the singular forms “a,” “an” and “the” are intended to include the plural forms as well, unless the context clearly indicates otherwise. Furthermore, to the extent that the terms “including,” “includes,” “having,” “has,” “with” or variants thereof are used in either the detailed description and/or the claims, such terms are intended to he inclusive in a manner similar to the term “comprising.” The transitional terms/phrases (and any grammatical variations thereof) “comprising,” “comprises,” “comprise,” “consisting essentially of,” “consists essentially of,” “consisting” and “consists” can be used interchangeably.

All patents, patent applications, provisional applications, and publications referred to or cited herein are incorporated by reference in their entirety, including all figures and tables, to the extent they are not inconsistent with the explicit teachings of this specification,

### REFERENCES

1. World Malaria Report 2014; Wodd Health Organization: 2014.

2. Bhatia, R., Rastogi, R. M., Ortega, L. Malaria successes and challenges in Asia. J. Vector Borne Dis. 50, 239-247 (2013).

3. Bloland, P. B. Drug resistance in malaria. Geneva: WHO; 2001.

4. Plowe, C. V. Malaria: Resistance nailed. Nature 505, 30-31 (2014).

5. Ariey, F. et aL A molecular marker of artemisinin-resistant Plasmodium falciparum malaria. Nature 505, 50-55 (2014).

6. Edgcornb, J. H. et al. Primaquine, SN 13272, a new curative agent in vivax malaria; a preliminary report. J Natl Malar Soc 9, 285-292 (1950).

7. Shanks. G. D. et al. A new primaquine analogue, tafenoquine (WR 238605), for prophylaxis against Plasmodium falciparum malaria. Clin. Infect. Dis. 33, 1968-1974 (2001).

8. Llands-Cuentas, A. et al. Tafenoquine plus chloroquine for the treatment and relapse prevention of Plasmodium vivax malaria (DETECTIVE): a multicentre, double-blind, randomised, phase 2b dose-selection study. Lancet 383, 1049-1058 (2014).

9. Ruwende, C, et al. Natural selection of hemi- and heterozygotes for G6PD deficiency in Africa by resistance to severe malaria. Nature 376, 246-249 (1995).

10. Vaccines, m.C.G.o. A research agenda for malaria eradication: drugs, PLoS Med. 8, e100040., (2011).

11. Burrows, J. et al. Designing the next generation of medicines for malaria control and eradication. Malar. J. 12, 187 (2013).

12. Alonso, P. L. et al. A Research Agenda to Underpin Malaria Eradication, PLoS Medi. 8, e1000406 (2011).

13. Charman, S. A. et al. Synthetic ozonide drug candidate OZ439 offers new hope for a single-dose cure of uncomplicated malaria. Proc. Natl. Acad. Sci. USA. 108, 4400-4405 (2011).

14. Gonzalez Cabrera, D. et al. Structure-activity relationship studies of orally active antimalarial 3,5-substituted 2-aminopyridines. J. Med Chem. 55, 11022-11030 (2012).

15. Younis, Y. et al. 3,5-Diaryl-2-aminopyridines as a novel class of orally active antimalarials demonstrating single dose cure in mice and clinical candidate potential. J. Med. Chem. 55, 3479-3487 (2012).

16. Jimenez-Diaz, M. B. et al. (+)-SJ733, a clinical candidate for malaria that acts through ATP4 to induce rapid host-mediated clearance of Plasmodium. Proc. Natl. Acad. Sci. U.S.A. 111, E5455-E5462 (2014).

17. Rottmann, M, et al. Spiroindolones, a potent compound class for the treatment of malaria. Science 329, 1175-1180 (2010).

18. Phillips, M. A. et al. A long-duration dihydroorotate dehydrogenase inhibitor (DSM265) for prevention and treatment of malaria. Sci. Transl. Med. 7, 296ra111. (2015).

19, Salzer, W., Timmler, H., Andersag, H. Uher einen neuen, gegen Vogelmalaria wirksamen Verbindungstypus. Chem. Ber. 81, 12-19 (1948).

20. Cross, R. M. et al. Endochin optimization: structure-activity and structure-property relationship studies of 3-substituted 2-methyl-4(1H)-quinolones with antimalarial activity. J. Med. Chem. 53, 7076-7094 (2010).

21. Winter, R. W. et al. Antimalarial quinolones: synthesis, potency, and mechanistic studies. Exp. Parasitol. 118, 487-497 (2008).

22. Winter, R. et al. Optimization of endochin-like quinolones for antimalarial activity. Exp. Parasitol. 127, 545-551 (2011).

23. Cross, R. M., Manetsch, R. Divergent route to access structurally diverse 4-quinolones via mono or sequential cross-couplings. J. Org. Chem. 75, 8654-8657 (2010).

24. Monastyrskyi, A., Namelikonda, N. K., Manetsch, R. Metal-Free Arylation of Ethyl Acetoacetate with Hypervalent Diaryliodonium Salts: An Immediate Access to Diverse 3-Aryl-4(1H)-Quinolones, J. Org. Chem. 80, 2513-2520 (2015).

25. Bueno, J. M. el al. Exploration of 4(1H)-pyridones as a novel family of potent antimalarial inhibitors of the plasmodial cytochrome bcl. Future Med. Chem. 4, 2311-2323 (2012).

26. Cross, R. M. et al. Optimization of 1,2,3,4-tetrahydroacridin-9(10H)-ones as antimalarials utilizing structure-activity and structure-property relationships. J. Med. Chem. 54, 4399-4426 (2011).

27. Cross, R. M. et al. Synthesis, antimalarial activity, and structure-activity relationship of 7-(2-phenoxyethoxy)-4(1H)-quinolones. J. Med. Chem. 54, 8321-8327 (2011).

28. Zhang, Y. et al. Lead optimization of 3-carboxyl-4(1H)-quinolones to deliver orally bioavailable antimalarials. J. Med. Chem. 55, 4205-4219 (2012).

29. Maignan, J. R. et al. ICI 56,780 Optimization: Structure-Activity Relationship Studies of 7-(2-Phenoxyethoxy)-4(1H)-quinolones with Antimalarial Activity. J. Med. Chem. 59, 6943-6960 (2016).

30. Biagini, G. A. et al. Generation of quinol one antimalarials targeting the Plasmodium falciparum mitochondrial respiratory chain for the treatment and prophylaxis of malaria. Proc. Natl. Acad. Sci. U. S. A. 109, 8298-8303 (2012).

31. Monastyrskyi, A., Kyle, D. E., Manetsch, R. 4(1H)-Pyridone and 4(1H)-Quinolone Derivatives as Antimalarials with Erythrocytic, Exoerythrocytic, and Transmission Blocking Activities. Curr. Top. Med. Chem, 14, 1693-1705 (2014).

32. Cross, R. M. et al. Orally Bioavailable 6-Chloro-7-methoxy-4(1H)-quinolones Efficacious against Multiple Stages of Plasmodium. J. Med Chem. 57, 8860-8879 (2014).

33. Nilsen, A. et al. Quinolone-3-diarylethers: a new class of antimalarial drug. Sci. Trcmsl. Med. 5, 177ra137 (2013).

34. Miley, G. P. et al. ELQ-300 prodrugs for enhanced delivery and single-dose cure of malaria. Antimicrob. Agents Chemother. 59, 5555-5560 (2015).

35. Potter, P. M., Watkins, R. M. Carboxylesterases—detoxifying enzymes and targets for drug therapy. Curr. Med. Chem. 13, 1045-1054 (2006).

36. Rautio, J. et al. Prodrugs: design and clinical applications. Nat. Rev. Drug Discov. 7, 255-270 (2008).

37. Nasiri, H. R., Bolte, M., Schwalbe, H. Tautomerism of 4-Hydroxy-4(1H) quinolon. Heterocycl. Commun. 12, 319-322 (2006).

38. Nilsen, A. et al. Discovery, Synthesis, and Optimization of Antimalarial 4(1H)-Quinolone-3-Diaryl ethers. J. Med. Chem. 57, 3818-3834 (2014).

39. Thomas, J. D., Sloan, K. B. Overcoming steric effects in the coupling reaction of alkyloxycarbonyloxymethyl (AOCOM) halides with phenols: an efficient synthesis of AOCOM phenolic prodrugs. Tetrahedron Lett. 48, 109-112 (2007).

40. Falkner, F. C. et al. Disposition of ampiroxicam, a prodrug of piroxicam, in man. Xenobiotica 20,645-652 (1990).

41. Gomes, P., Vale, N., Moreira, R. Cyclization-activated prodrugs. Molecules 12, 2484-2506 (2007).

42. Babij, N. R et al. NMR Chemical Shifts of Trace Impurities: Industrially Preferred Solvents Used in Process and Green Chemistry. Org. Process Res. Dev. 20, 661-667 (2016).

43. Chittiboyina, A. G. et al. Design, synthesis, and docking studies of novel telmisartan-glitazone hybrid analogs for the treatment of metabolic syndrome. Med. Chem. Res. 18, 589-610 (2009).

44. Inoue, Y. el al. Fluorescent molecularly imprinted polymer thin films for specific protein detection prepared with dansyl ethylenediamine-conjugated O-acryloyl L-hydroxyproline. Biosens. Bioelectron. 48, 113-119 (2013).

45. Heck, H. D.; Casanova-Schmitz, M.; Dodd, P. B.; Schachter, E. N.; Witek, T. J.; Tosun, T. Formaldehyde (CH₂O) concentrations in the blood of humans and Fischer-344 rats exposed to CH₂O under controlled conditions. Am. Ind. Hyg. Assoc. J. 46, 1-3 (1985).

Rietbrock, N. Kinetics and pathways of methanol metabolism. Nounyn. Schmiedebergs Arch. Exp. Pathol. Pharmakol. 263, 88-105 (1969).

Owen, B. A.; Dudney, C. S.; Tan, E. L.; Easterly, C. E. Formaldehyde in Drinking—Water—Comparative Hazard Evaluation and an Approach to Regulation. Regul. Toxicol. Phormacol. 11, 220-236 (1990).

Casciere, T. C.; Clary, J. J. Formaldehyde—oral toxicity assessment, Comments Toxicol. 4, 295-304 (1992).

Cerebyx U.S. Physician Prescribing Information; Pfizer Labs: New York 2016.

Nilsen, A.; LaCrue, A. N.; White, K. L.; Forquer, I. P.; Cross, R. M.; Marfurt, J.; Mather, M. W.; Delves, M. J.; Shackieford, D. M.; Saenz, F. E.; Morrisey, J. M.; Steuten, J.; Mutka, T.; Li, Y.; Witjanata, G.; Ryan, E.; Duffy, S.; Kelly, J. X.; Sebayang, B. F.; Zeeman, A. M.; Noviyanti, R.; Sinden, R. E.; Kocken, C. H.; Price, R. N.; Avery, V. M.; Angulo-Barturen, I.; Jimenez-Diaz, M. B.; Ferrer, S.; Herreros, E.; Sanz, L. M.; Gamo, F. J.; Bathurst, I; Burrows, J. N.; Siegl, P.; Guy, R. K.; Winter, R. W.; Vaidya, A. B.; Charman, S. A.; Kyle; D. E.; Tvlanetsch, R.; Riscoe, M. K. Quinolone-3-diarylethers: a new class of antimalarial drug. Sci. Transl. Med. 5, 177ra37 (2013).

1. A compound or a salt thereof, the compound comprising a Formula (I): 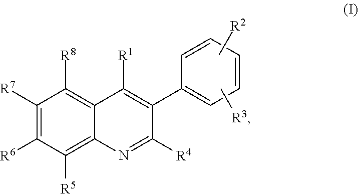
wherein R¹ is selected from H, F, Cl, Br, I, CN, CH₃, CF₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, carbamate, alkyltriphenylphosphonium, 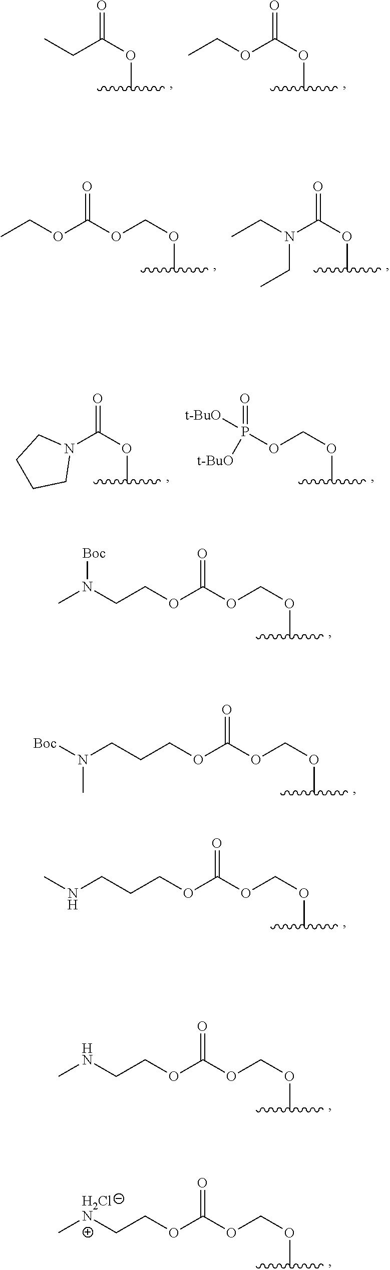 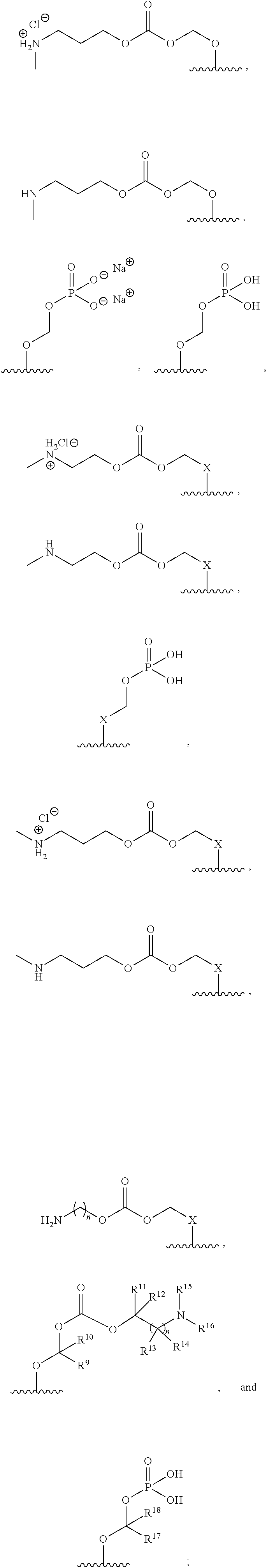
wherein R², R³, R⁴, R⁵, R⁶, R⁷, R⁸, R⁹, R¹⁹, R¹¹, R¹², R¹³, R¹⁴, R¹⁵, R¹⁶, R¹⁷, and R¹⁸ are independently selected from H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium, and 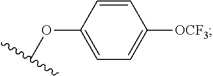
wherein X is selected from NH, NR¹⁹, oxygen, sulfur, and selenium, wherein R¹⁹ is selected from the group H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium; and
wherein n is 1, 2, 3, or 4.

2. The compound of claim 1, wherein the compound comprises a formula:

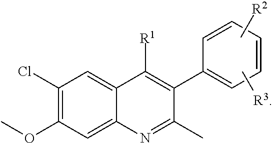

3. The compound of claim 1, wherein the compound comprises a formula:

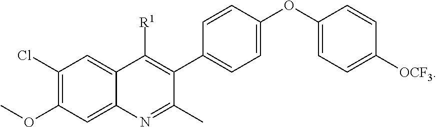

4. The compound of claim 3, wherein the compound comprises a formula:

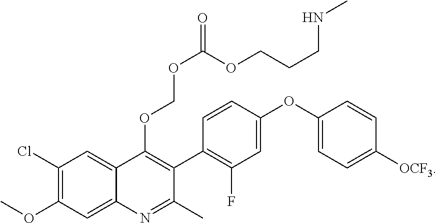 [SMILES: CNCCCOC(=O)OCOc1c(-c2ccc(Oc3ccc(OC(F)(F)F)cc3)cc2F)c(C)nc2cc(OC)c(Cl)cc12]

5. The compound of claim 3, wherein the compound comprises a formula:

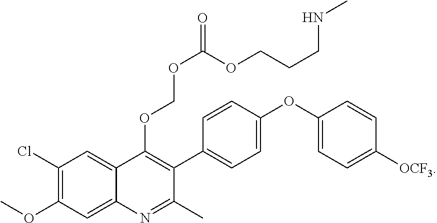 [SMILES: CNCCCOC(=O)OCOc1c(-c2ccc(Oc3ccc(OC(F)(F)F)cc3)cc2)c(C)nc2cc(OC)c(Cl)cc12]

6. A composition comprising:
a compound or a salt thereof, the compound comprising a Formula (I): 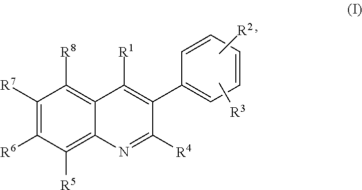
wherein R¹ is selected from H, F, Cl, Br, I, CN, CHs, CF₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, carbamate, alkyltriphenylphosphonium, 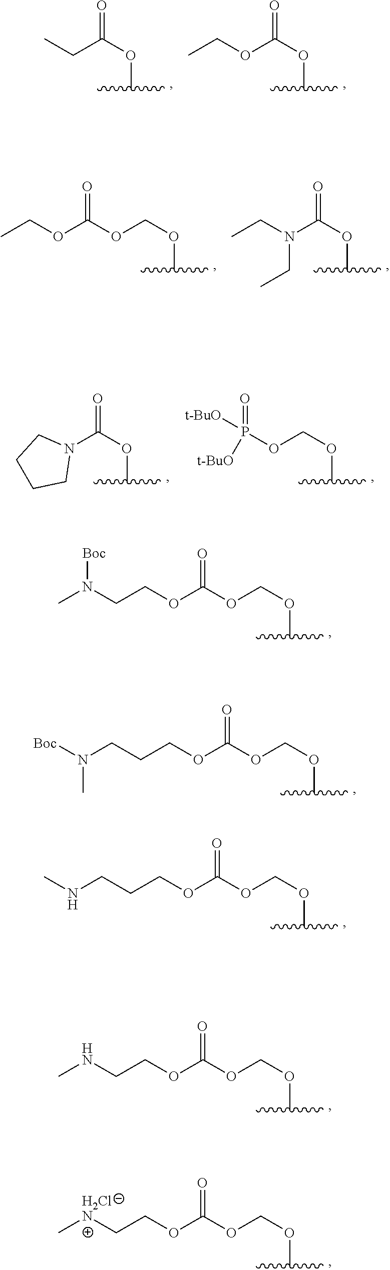 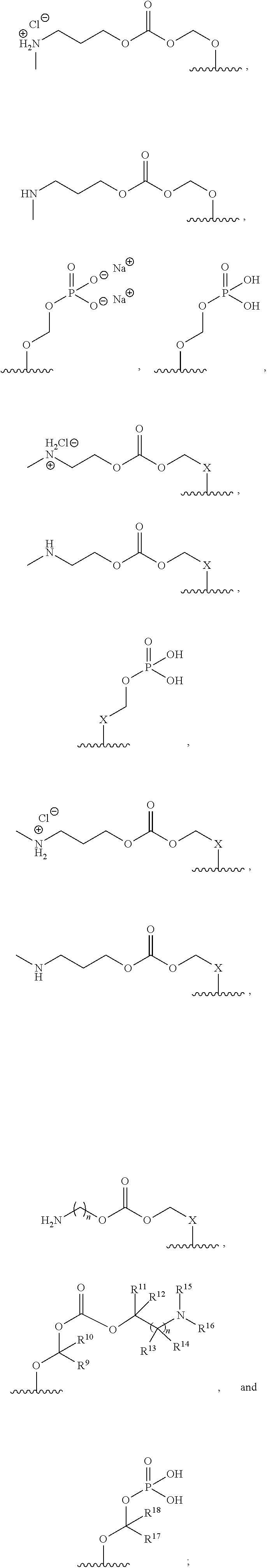
wherein R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently selected from H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium, and 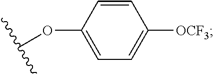
wherein X is selected from NH, NR¹⁹, oxygen, sulfur, and selenium, wherein R¹⁹ is selected from the group H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium: and
wherein n is 1, 2, 3, or 4; and
a pharmaceutically acceptable carrier.

7.-8. (canceled)

9. The composition of claim 6, wherein the compound comprises a formula:

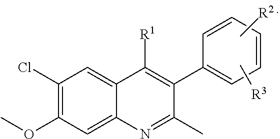

10. The composition of claim 6, wherein the compound comprises a formula:

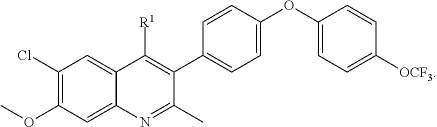

11. The compound of claim 10, wherein the compound comprises a formula:

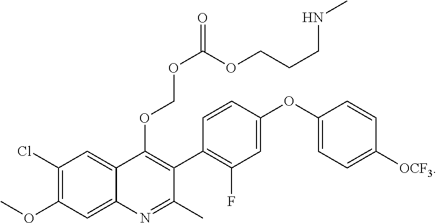 [SMILES: CNCCCOC(=O)OCOc1c(-c2ccc(Oc3ccc(OC(F)(F)F)cc3)cc2F)c(C)nc2cc(OC)c(Cl)cc12]

12. The composition of claim 10, wherein the compound comprises a formula:

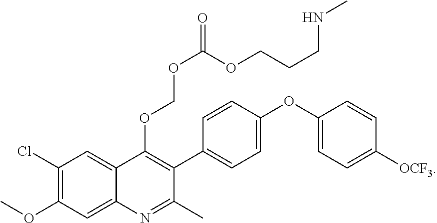 [SMILES: CNCCCOC(=O)OCOc1c(-c2ccc(Oc3ccc(OC(F)(F)F)cc3)cc2)c(C)nc2cc(OC)c(Cl)cc12]

13. A method of treating malaria in a subject in need thereof, the method comprising:
administering an amount of a compound to the subject in need thereof, the compound comprising a Formula (I): 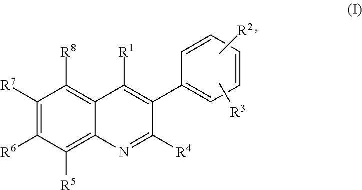
wherein R¹ is selected from H, F, Cl, Br, I, CN, CHs, CF₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, carbamate, alkyltriphenylphosphonium, 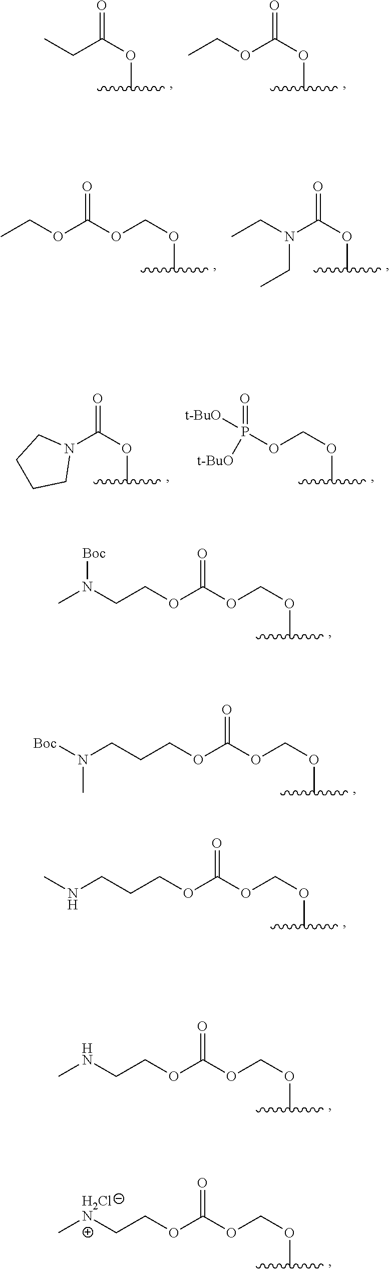 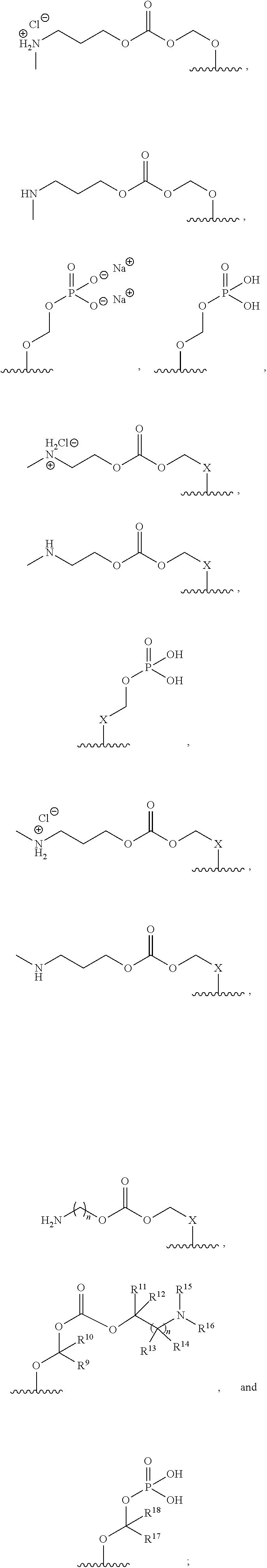
wherein R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently selected from H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium, and 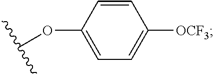
wherein X is selected from NH, NR¹⁹, oxygen, sulfur, and selenium, wherein R¹⁹ is selected from the group H, F, Cl, Br, I, CN, CH₃, CF₃, OCH₃, alkyl, halogenated alkyl, heteroalkyl, alkenyl, alkynyl, aryl, arylalkyl, aryloxy, arylalkoxy, heteroalkyl, heteroaryl, heterocyclyl, cycloalkyl, cycloalkenyl, cycloalkynyl, hydroxyl, hydroxyalkyl, alkoxy, alkoxyalkyl, amino, aminoalkyl, alkylamino, diarylamino, dialkylamino, arylamino, alkylarylamino, acyl, acylamino, thiol, thioalkyl, alkylthio, acyloxy, nitro, oxo, carbamoyl, trifluoromethyl, phenoxy, benzyloxy, phosphonic acid, phosphate ester, sulfonic acid (—SO₃H), sulfonate ester, sulfonamide, and carbamate, alkyltriphenylphosphonium; and
wherein n is 1, 2, 3, or 4.

14. The method of claim 13, wherein the compound comprises a formula:

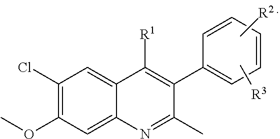

15. The method of claim 13, wherein the compound comprises a formula:

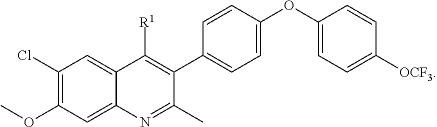

16. The method of claim 15, wherein the compound comprises a formula:

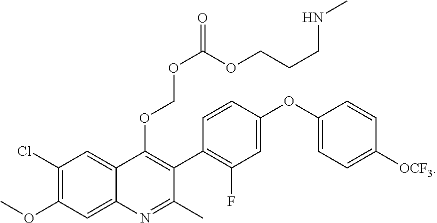 [SMILES: CNCCCOC(=O)OCOc1c(-c2ccc(Oc3ccc(OC(F)(F)F)cc3)cc2F)c(C)nc2cc(OC)c(Cl)cc12]

17. The method of claim 15, wherein the compound comprises a formula:

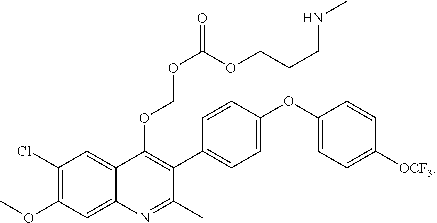 [SMILES: CNCCCOC(=O)OCOc1c(-c2ccc(Oc3ccc(OC(F)(F)F)cc3)cc2)c(C)nc2cc(OC)c(Cl)cc12]

18. The method of claim 13, wherein the compound is formulated as a composition comprising the compound and a pharmaceutically acceptable carrier.

19. The method of claim 13, wherein the amount of the compound ranges from about 1 mg/kg to about 30 mg/kg.
